(19) United States
(12) Patent Application Publication
(10) Pub. No.: US 2007/0249705 A1
(43) Pub. Date: Oct. 25, 2007

(54) N-(1-(2,3-Dihydro-1H-Inden-5Yl)-2-Oxo-3-Pyrrolidinyl)-Sulfonamide Derivatives for Use as Factor Xa Inhibitors

(76) Inventors: John David Harling, Stevenage (GB); Savvas Kleanthous, Stevenage (GB); Nigel Stephen Watson, Stevenage (GB); Robert Ian West, Stevenage (GB); Robert John Young, Stevenage (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 11/574,694

(22) PCT Filed: Sep. 2, 2005
(86) PCT No.: PCT/EP05/09517

§ 371 Date: Mar. 5, 2007

(30) Foreign Application Priority Data
Sep. 6, 2004 (GB) 0419744.8

(51) Int. Cl. A61K 31/402; A61K 31/4025; A61K 31/404; A61P 7/02; C07D 207/273; C07D 403/12; C07D 403/14; C07D 405/12; C07D 409/12

(52) U.S. Cl. 514/422; 514/424; 548/518; 548/525; 548/527; 548/550

(57) ABSTRACT

The invention relates to at least one chemical entity chosen from compounds of formula (I): 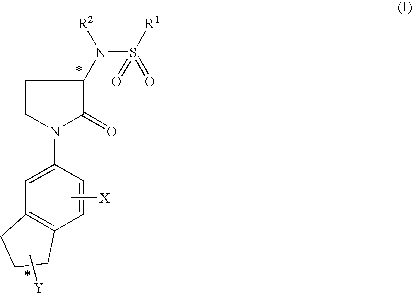 and pharmaceutically acceptable derivative(s) thereof. The invention also relates to processes for the preparation of compound(s) of formula (I), pharmaceutical compositions containing compound(s) of formula (I) and to the use of compound(s) of formula (I) in medicine, particularly in the amelioration of a clinical condition for which a Factor Xa inhibitor is indicated.

### FIELD OF THE INVENTION

The present invention relates to a novel class of chemical compounds, to processes for their preparation, to pharmaceutical compositions containing them and to their use in medicine, particularly use in the amelioration of a clinical condition for which a Factor Xa inhibitor is indicated.

### BACKGROUND OF THE INVENTION

Factor Xa is a member of the trypsin-like serine protease class of enzymes. It is a key enzyme in the coagulation cascade. A one-to-one binding of Factors Xa and Va with calcium ions and phospholipid converts prothrombin into thrombin. Thrombin plays a central role in the mechanism of blood coagulation by converting the soluble plasma protein, fibrinogen, into insoluble fibrin. The insoluble fibrin matrix is required for the stabilisation of the primary hemostatic plug. Many significant disease states are related to abnormal hemostasis. With respect to the coronary arterial vasculature, abnormal thrombus formation due to the rupture of an established atherosclerotic plaque is the major cause of acute myocardial infarction and unstable angina. Both treatment of an occlusive coronary thrombus by thrombolytic therapy and percutaneous transluminal coronary angioplasty (PTCA) are often accompanied by an acute thrombotic reclosure of the affected vessel which requires immediate resolution. With respect to the venous vasculature, a high percentage of patients undergoing major surgery in the lower extremities or the abdominal area suffer from thrombus formation in the venous vasculature which can result in reduced blood flow to the affected extremity and a pre-disposition to pulmonary embolism. Disseminated intravascular coagulopathy commonly occurs within both vascular systems during septic shock, certain viral infections and cancer and is characterised by the rapid consumption of coagulation factors and systemic coagulation which results in the formation of life-threatening thrombi occurring throughout the vasculature leading to widespread organ failure. Beyond its direct role in the formation of fibrin rich blood clots, thrombin has been reported to have profound bioregulatory effects on a number of cellular components within the vasculature and blood, (Shuman, M. A., Ann. NY Acad. Sci., 405: 349 (1986)).

A Factor Xa inhibitor may be useful in the treatment of acute vascular diseases such as acute coronary syndromes (for example primary and secondary prevention of myocardial infarction and unstable angina and treatment of prothrombotic sequalae associated with myocardial infarction or heart failure), thromboembolism, acute vessel closure associated with thrombolytic therapy and percutaneous transluminal coronary angioplasty, transient ischemic attacks, pulmonary embolism, deep vein thrombosis, peripheral arterial occlusion, prevention of vessel luminal narrowing (restenosis), and the prevention of thromboembolic events associated with atrial fibrillation, e.g. stroke. Factor Xa inhibitors may also be useful in preventing thrombosis and complications in patients genetically predisposed to arterial thrombosis or venous thrombosis and patients that have a disease-associated predisposition to thrombosis (e.g. type 2 diabetics). Thrombin has been reported to contribute to lung fibroblast proliferation, thus, Factor Xa inhibitors could be useful for the treatment of some pulmonary fibrotic diseases. Factor Xa inhibitors could also be useful in the treatment of tumour metastasis, by suppressing coagulation and thus preventing fibrin deposition and its concomitant facilitation of metastasis. A Factor Xa inhibitor may also have utility as an anti-inflammatory agent through its inhibition of FXa mediated activation of protease-activated receptors (PAR 1-4). A Factor Xa inhibitor may also have utility as an anti-atherosclerotic agent through the suppression of platelet-activation. Thrombin can induce neurite retraction and thus Factor Xa inhibitors may have potential in neurogenerative diseases such as Parkinson's and Alzheimer's disease. Factor Xa inhibitors may also have utility as anticoagulant agents in connection with the preparation, storage, fractionation or use of whole blood. They have also been reported for use in conjunction with thrombolytic agents, thus permitting the use of a lower dose of thrombolytic agent.

### DESCRIPTION OF THE INVENTION

The present invention provides at least one chemical entity chosen from compounds of formula (I): 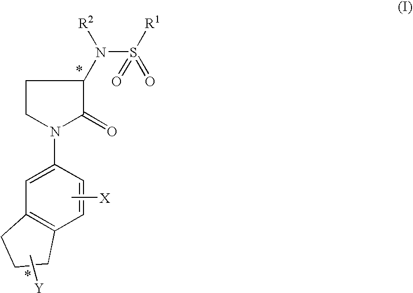
wherein:
R¹ represents a group selected from: 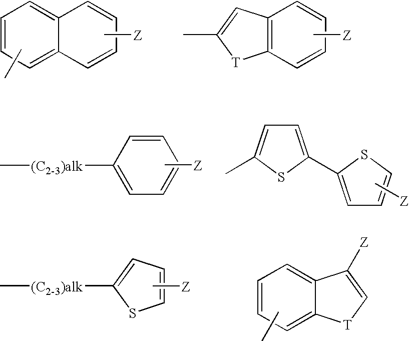
each ring of which optionally contains a further heteroatom N,
Z represents an optional substituent halogen,
alk represents alkylene or alkenylene,
T represents S, O or NH;
R² represents hydrogen, —C₁₋₆alkyl, —C₁₋₃alkylCONR^{a}R^{b}, —C₁₋₃alkylCO₂C₁₋₄alkyl, —CO₂C₁₋₄alkyl or —C₁₋₃alkylCO₂H;
R^{a} and R^{b} independently represent hydrogen, —C₁₋₆alkyl, or together with the N atom to which they are bonded form a 5-, 6- or 7-membered non-aromatic heterocyclic ring optionally containing an additional heteroatom selected from O, N and S, optionally substituted by C₁₋₄alkyl, and optionally the S heteroatom is substituted by O, i.e. represents S(O)ₙ;
n represents 0-2;
X represents an optional substituent on the indane ring selected from: halogen, —C₁₋₄alkyl, —C₂₋₄alkenyl and —CF₃;
Y represents a group —(CH₂)ₘNR^{c}R^{d} substituted on the non-aromatic portion of the indane ring;
R^{c} and R^{d} independently represent hydrogen, —C₁₋₆alkyl, —C₁₋₄alkylOH, or together with the atom to which they are bonded form a 4-, 5-, 6- or 7-membered non-aromatic heterocyclic ring, the 5-, 6- or 7-membered non-aromatic heterocyclic ring optionally containing an additional heteroatom selected from O, N or S, and the 4-, 5-, 6- or 7-membered non-aromatic heterocyclic ring optionally substituted by C₁₋₄alkyl or halogen, e.g. fluorine;
m represents 0-2;
and pharmaceutically acceptable derivative(s) thereof.

Further aspects of the invention are:
A pharmaceutical composition comprising a compound of the invention together with a pharmaceutical carrier and/or excipient.
A compound of the invention for use in therapy.
Use of a compound of the invention for the manufacture of a medicament for the treatment of a patient suffering from a condition susceptible to amelioration by a Factor Xa inhibitor.
A method of treating a patient suffering from a condition susceptible to amelioration by a Factor Xa inhibitor comprising administering a therapeutically effective amount of a compound of the invention.

The present invention also provides compounds of formula (I): 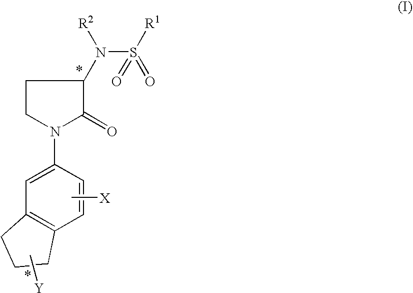
wherein:
R¹ represents a group selected from: 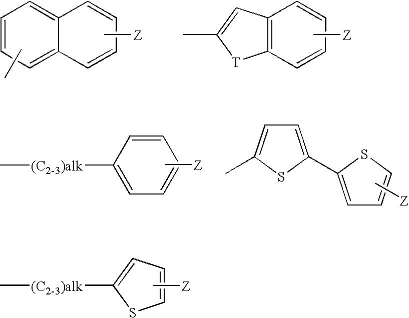
each ring of which optionally contains a further heteroatom N,
Z represents an optional substituent halogen,
alk represents alkylene or alkenylene,
T represents S, O or NH;
R² represents hydrogen, —C₁₋₆alkyl, —C₁₋₃alkylCNR^{a}R^{b}, alkylC₂C₁₋₄alkyl, —CO₂C₁₋₄alkyl or —C₁₋₃alkylCO₂H;
R^{a} and R^{b} independently represent hydrogen, —C₁₋₆alkyl, or together with the N atom to which they are bonded form a 5-, 6- or 7-membered non-aromatic heterocyclic ring optionally containing an additional heteroatom selected from O, N and S, optionally substituted by C₁₋₄alkyl, and optionally the S heteroatom is substituted by O, i.e. represents S(O)ₙ;
n represents 0-2;
X represents an optional substituent on the indane ring selected from: halogen, —C₁₋₄alkyl, —C₂₋₄alkenyl and —CF₃;
Y represents a group —(CH₂)ₘNR^{c}R^{d} substituted on the non-aromatic portion of the indane ring;
R^{c} and R^{d} independently represent hydrogen, —C₁₋₆alkyl, —C₁₋₄alkylOH, or together with the N atom to which they are bonded form a 4-, 5-, 6- or 7-membered non-aromatic heterocyclic ring, the 5-, 6- or 7-membered non-aromatic heterocyclic ring optionally containing an additional heteroatom selected from O, N or S, and the 4-, 5-, 6- or 7-membered non-aromatic heterocyclic ring optionally substituted by C₁₋₄alkyl or halogen, e.g. fluorine;
m represents 0-2;
and pharmaceutically acceptable derivative(s) thereof.

In one aspect of the invention, R¹ represents a group selected from: 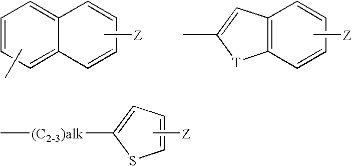
each ring of which optionally contains a further heteroatom N,
Z represents an optional substituent halogen,
alk represents alkylene or alkenylene,
T represents S, O or NH.

In another aspect, R¹ represents a group selected from: 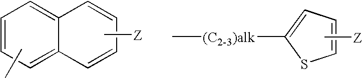
each ring of which optionally contains a further heteroatom N,
Z represents an optional substituent halogen,
alk represents alkylene or alkenylene.

In another aspect, R¹ represents a group selected from: 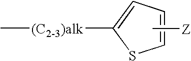
Z represents an optional substituent halogen,
alk represents alkylene or alkenylene.

Alternatively, R¹ represents a group selected from: 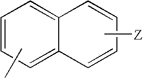
Z represents an optional substituent halogen.

In one aspect of the invention, T represents S or N. In another aspect of the invention, T represents S.

In one aspect of the invention, R² represents hydrogen or —C₁₋₆alkyl. In another aspect of the invention, R² represents hydrogen or methyl. In another aspect of the invention, R² represents hydrogen.

In one aspect of the invention, R^{a} and R^{b} independently represent hydrogen or —C₁₋₆alkyl.

In one aspect of the invention, n represents 0-2.

In one aspect of the invention, X represents an optional substituent selected from: halogen and —C₁₋₄alkyl. In another aspect, X is absent.

In one aspect of the invention, Y represents a group —NR^{c}R^{d}. Y may be substituted at any position on the non-aromatic portion of the indane ring but is preferably substituted as shown below:

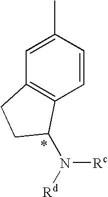

In one aspect of the invention, R^{c} and R^{d} independently represent hydrogen or —C₁₋₆alkyl. In another aspect, R^{c} and R^{d} both represent methyl. In another aspect, R^{c} represents hydrogen and R^{d} represents methyl.

It is to be understood that the present invention covers all combinations of the various aspects of the invention described herein above.

As used herein, the term “alkyl” means both straight and branched chain saturated hydrocarbon groups. Examples of alkyl groups include methyl (—CH₃), ethyl (—C₂H₅), propyl (—C₃H₇) and butyl (—C₄H₉).

As used herein, the term “alkylene” means both straight and branched chain saturated hydrocarbon linker groups. Examples of alkylene groups include methylene (—CH₂—), ethylene (—CH₂CH₂—) and propylene (—CH₂CH₂CH₂—).

As used herein, the term “alkenylene” means both straight and branched chain unsaturated hydrocarbon linker groups, wherein the unsaturation is present only as double bonds. Examples of alkenylene groups includes ethenylene (—CH═CH—) and propenylene (—CH₂—CH═CH—).

As used herein, the term “non-aromatic heterocyclic ring” means optionally substituted rings containing one or more heteroatoms selected from: nitrogen, sulphur and oxygen atoms. Examples of 4-membered rings include azetidinyl. Examples of 5-membered groups include pyrrolidinyl. Examples of 6-membered rings include piperidinyl and morpholinyl. Examples of 7-membered rings include hexamethyleneiminyl.

As used herein, the term “halogen” means an atom selected from fluorine, chlorine, bromine and iodine.

As used herein, the term “pharmaceutically acceptable” means a compound which is suitable for pharmaceutical use.

As used herein, the term “pharmaceutically acceptable derivative”, means any pharmaceutically acceptable salt, solvate, or prodrug e.g. ester or carbamate, or salt or solvate of such a prodrug, of a compound of formula (I), which upon administration to the recipient is capable of providing (directly or indirectly) a compound of formula (I), or an active metabolite or residue thereof. Exemplary pharmaceutically acceptable derivatives are salts, solvates, esters and carbamates. More exemplary pharmaceutically acceptable derivatives are salts, solvates and esters. Even more exemplary pharmaceutically acceptable derivatives are salts and solvates.

Suitable salts according to the invention may include those formed with both organic and inorganic acids and bases. Pharmaceutically acceptable acid addition salts include those formed from mineral acids such as: hydrochloric, hydrobromic, sulphuric, phosphoric, acid; and organic acids such as: citric, tartaric, lactic, pyruvic, acetic, trifluoroacetic, succinic, oxalic, formic, fumaric, maleic, oxaloacetic, methanesulphonic, ethanesulphonic, p-toluenesulphonic, benzenesulphonic and isethionic acids. Exemplary pharmaceutically acceptable salts include those formed from hydrochloric, trifluoroacetic and formic acids. Thus, in one aspect of the invention pharmaceutically acceptable salts are formic acid salts.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compound of formula (I) are within the scope of the invention.

Salts and solvates of compounds of formula (I) which are suitable for use in medicine may be those wherein the counterion or associated solvent is pharmaceutically acceptable. However, salts and solvates having non-pharmaceutically acceptable counterions or associated solvents are within the scope of the present invention, for example, for use as intermediates in the preparation of other compounds of formula (I) and their pharmaceutically acceptable salts and solvates.

The compounds of formula (I) contain chiral (asymmetric) centres (as indicated by the symbol *). The individual stereoisomers (enantiomers and diastereoisomers) and mixtures of these are within the scope of the present invention. In one aspect of the invention, the stereochemistry is (S) at the 3-position on the 2-oxopyrrolidine ring (as indicated by the symbol * in formula (I)).

It will also be appreciated that compounds of the invention which exist as polymorphs, and mixtures thereof, are within the scope of the present invention.

As used herein, the term “prodrug” means a compound which is converted within the body, e.g. by hydrolysis in the blood, into its active form that has medical effects. Pharmaceutically acceptable prodrugs are described in T. Higuchi and V. Stella, Prodrugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and in D. Fleisher, S. Ramon and H. Barbra “Improved oral drug delivery: solubility limitations overcome by the use of prodrugs”, Advanced Drug Delivery Reviews (1996) 19(2) 115-130, each of which are incorporated herein by reference.

Prodrugs are any covalently bonded carriers that release a compound of structure (I) in vivo when such prodrug is administered to a patient. Prodrugs are generally prepared by modifying functional groups in a way such that the modification is cleaved in vivo yielding the parent compound. Prodrugs may include, for example, compounds of this invention wherein hydroxyl or amine groups are bonded to any group that, when administered to a patient, cleaves to form the hydroxyl or amine groups.

Esters may be active in their own right and/or be hydrolysable under in vivo conditions in the human body. Suitable pharmaceutically acceptable in vivo hydrolysable ester groups include those which break down readily in the human body to leave the parent acid or its salt. An ester may be formed at a carboxylic acid (—COOH) group or a hydroxyl (—OH) group, by methods well known in the art involving reaction with the corresponding alcohol, acid, acid chloride, anhydride, or amide. For example, esters may be C₁₋₆alkyl esters, e.g. methyl esters, ethyl esters, and the like.

As used herein, the term “compounds of the invention” means both the compounds according to formula I and the pharmaceutically acceptable derivatives thereof. The terms “a compound of the invention” and “chemical entity” also appear herein and refer to both a compound according to formula I and its pharmaceutically acceptable derivatives.

In one aspect, chemical entities useful in the present invention may be at least one chemical entity selected from the list:
6-Chloro-N-{1-[1-(dimethylamino)-2,3-dihydro-1H-inden-5-yl]-2-oxo-3-pyrrolidinyl}-2-naphthalenesulfonamide;
(E)-2-(5-Chloro-2-thienyl)-N-{1-[1-(dimethylamino)-2,3-dihydro-1H-inden-5-yl]-2-oxo-3-pyrrolidinyl}ethenesulfonamide;
6-Chloro-N-{1-[1-(dimethylamino)-2,3-dihydro-1H-inden-5-yl]-2-oxo-3-pyrrolidinyl}-1-benzothiophene-2-sulfonamide;
6-Chloro-N-{(3S)-1-[-(dimethylamino)-2,3-dihydro-1H-inden-5-yl]-2-oxo-3-pyrrolidinyl-2-naphthalenesulfonamide;
3-Chloro-N-{(3S)-1-[1-(dimethylamino)-2,3-dihydro-1H-inden-5-yl]-2-oxo-3-pyrrolidinyl}-1H-indole-6-sulfonamide;
2-(5-Chloro-2-thienyl)-N-{(3S)-1-[1-(dimethylamino)-2,3-dihydro-1H-inden-5-yl]-2-oxo-3-pyrrolidinyl}ethanesulfonamide;
6-Chloro-N-{(3S)-1-[1-(methylamino)-2,3-dihydro-1H-inden-5-yl]-2-oxo-3-pyrrolidinyl}-2-naphthalenesulfonamide;
(E)-2-(5-Chloro-2-thienyl)-N-{(3S)-1-[1-(methylamino)-2,3-dihydro-1H-inden-5-yl]-2-oxo-3-pyrrolidinyl}ethenesulfonamide;
2-(5-Chloro-2-thienyl)-N-{(3S)-1-[1-(methylamino)-2,3-dihydro-1H-inden-5-yl]-2-oxo-3-pyrrolidinyl}ethanesulfonamide;
3-Chloro-N-{(3S)-1-[1-(methylamino)-2,3-dihydro-1H-inden-5-yl]-2-oxo-3-pyrrolidinyl}-1H-indole-6-sulfonamide;
N-[(3S)-1-(1-Amino-2,3-dihydro-1H-inden-5-yl)-2-oxo-3-pyrrolidinyl]-6-chloro-2-naphthalenesulfonamide; and
6-Chloro-N-methyl-N-{(3S)-1-[1-(methylamino)-2,3-dihydro-1H-inden-5-yl]-2-oxo-3-pyrrolidinyl}-2-naphthalenesulfonamide.

Compounds of the invention may show advantageous properties, they may be more efficacious, may show greater selectivity, may have fewer side effects, may have a longer duration of action, may be more bioavailable by the preferred route, or may have other more desirable properties than similar known compounds.

The compounds of formula (I) are Factor Xa inhibitors and as such are useful in the treatment of clinical conditions susceptible to amelioration by administration of a Factor Xa inhibitor. Such conditions may include acute vascular diseases such as acute coronary syndromes (for example primary and secondary prevention of myocardial infarction and unstable angina and treatment of prothrombotic sequalae associated with myocardial infarction or heart failure), thromboembolism, acute vessel closure associated with thrombolytic therapy and percutaneous transluminal coronary angioplasty (PTCA), transient ischemic attacks, pulmonary embolism, deep vein thrombosis, peripheral arterial occlusion, prevention of vessel luminal narrowing (restenosis), and the prevention of thromboembolic events associated with atrial fibrillation, e.g. stroke; in preventing thrombosis and complications in patients genetically predisposed to arterial thrombosis or venous thrombosis and patients that have a disease-associated predisposition to thrombosis (e.g. type 2 diabetics); the treatment of pulmonary fibrosis; the treatment of tumour metastasis; inflammation; atherosclerosis; neurogenerative disease such as Parkinson's and Alzheimer's diseases; Kasabach Merritt Syndrome; Haemolytic uremic syndrome; endothelial dysfunction; as anti-coagulants for extracorporeal blood in for example, dialysis, blood filtration, bypass, and blood product storage; and in the coating of invasive devices such as prostheses, artificial valves and catheters in reducing the risk of thrombus formation.

Accordingly, one aspect of the present invention provides at least one chemical entity chosen from compounds of formula (I) and pharmaceutically acceptable derivative(s) thereof for use in medical therapy, for example, for use in the amelioration of a clinical condition in a mammal, including a human, for which a Factor Xa inhibitor is indicated.

In another aspect, the invention provides a method for the treatment and/or prophylaxis of a condition susceptible to amelioration by a Factor Xa inhibitor in a mammal, including a human, which method comprises administering to the subject an effective amount of at least one chemical entity chose from compounds of formula (I) and pharmaceutically acceptable derivative(s) thereof.

In another aspect, the present invention provides the use of at least one chemical entity chosen from compounds of formula (I) and pharmaceutically acceptable derivative(s) thereof, for the manufacture of a medicament for the treatment and/or prophylaxis of a condition susceptible to amelioration by a Factor Xa inhibitor.

In one aspect of the invention, the condition susceptible to amelioration by a Factor Xa inhibitor is selected from treatment of acute vascular diseases such as acute coronary syndromes (for example primary and secondary prevention of myocardial infarction and unstable angina and treatment of prothrombotic sequalae associated with myocardial infarction or heart failure), thromboembolism, acute vessel closure associated with thrombolytic therapy and percutaneous transluminal coronary angioplasty, transient ischemic attacks, pulmonary embolism, deep vein thrombosis, peripheral arterial occlusion, prevention of vessel luminal narrowing (restenosis), and the prevention of thromboembolic events associated with atrial fibrillation, e.g. stroke.

In another aspect, the condition susceptible to amelioration by a Factor Xa inhibitor is selected from acute coronary syndromes (for example primary and secondary prevention of myocardial infarction and unstable angina and treatment of prothrombotic sequalae associated with myocardial infarction or heart failure), pulmonary embolism, deep vein thrombosis and the prevention of thromboembolic events associated with atrial fibrillation, e.g. stroke.

It will be appreciated that reference to treatment includes acute treatment or prophylaxis as well as the alleviation of established symptoms.

Within the context of the present invention, the terms describing the indications used herein are classified in the The Merck Manual of Diagnosis and Therapy, 17^{th} Edition and/or the International Classification of Diseases, 10th Edition (ICD-10). The various subtypes of the disorders mentioned herein are contemplated as part of the present invention.

While it is possible that, for use in therapy, a compound of the present invention may be administered as the raw chemical, the active ingredient may also be presented as a pharmaceutical formulation.

In a further aspect, the invention provides a pharmaceutical composition comprising at least one chemical entity chosen from compounds of formula (I) and pharmaceutically acceptable derivative(s) thereof in association with at least one pharmaceutically acceptable carrier and/or excipient. The carrier and/or excipient must be “acceptable” in the sense of being compatible with the other ingredients of the formulation and not deleterious to the recipient thereof.

In another aspect, the invention provides a pharmaceutical composition comprising, as active ingredient, at least one chemical entity chosen from compounds of formula (I) and pharmaceutically acceptable derivative(s) thereof in association with a pharmaceutically acceptable carrier and/or excipient for use in therapy, and for example in the treatment of human or animal subjects suffering from a condition susceptible to amelioration by a Factor Xa inhibitor.

There is further provided by the present invention a process of preparing a pharmaceutical composition, which process comprises mixing at least one chemical entity chosen from compounds of formula (I) and pharmaceutically acceptable derivative(s) thereof, together with at least one pharmaceutically acceptable carrier and/or excipient.

The compounds for use according to the present invention may be formulated for oral, buccal, parenteral, topical, rectal or transdermal administration or in a form suitable for administration by inhalation or insufflation (either through the mouth or the nose).

For oral administration, the pharmaceutical compositions may take the form of, for example, tablets or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycollate); or wetting agents (e.g. sodium lauryl sulphate). The tablets may be coated by methods well known in the art. Liquid preparations for oral administration may take the form of, for example, solutions, syrups or suspensions or they may be presented as a dry product for constitution with water or other suitable vehicles before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. almond oil, oily esters, ethyl alcohol or fractionated vegetable oils); and preservatives (e.g. methyl or propyl-p-hydroxybenzoates or sorbic acid). The preparations may also contain buffer salts, flavouring, colouring and sweetening agents as appropriate.

Preparations for oral administration may be suitably formulated to give controlled/extended release of the active compound.

For buccal administration the compositions may take the form of tablets or lozenges formulated in a conventional manner.

The compounds according to the present invention may be formulated for parenteral administration by injection, e.g. by bolus injection or continuous infusion. Formulations for injection may be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use.

The compounds according to the present invention may be formulated for topical administration by insufflation and inhalation. Examples of types of preparation for topical administration include sprays and aerosols for use in an inhaler or insufflator.

Powders for external application may be formed with the aid of any suitable powder base, for example, lactose, talc or starch. Spray compositions may be formulated as aqueous solutions or suspensions or as aerosols delivered from pressurised packs, such as metered dose inhalers, with the use of a suitable propellant.

The compounds according to the present invention may also be formulated in rectal compositions such as suppositories or retention enemas, e.g. containing conventional suppository bases such as cocoa butter or other glycerides.

In addition to the formulations described previously, the compounds may also be formulated as a depot preparation. Such long acting formulations may be administered by implantation (for example subcutaneously, transcutaneously or intramuscularly) or by intramuscular injection. Thus, for example, the compounds according to the present invention may be formulated with suitable polymeric or hydrophobic materials (for example as an emulsion in an acceptable oil) or ion exchange resins or as sparingly soluble derivatives, for example, as a sparingly soluble salt.

A proposed dose of the compounds according to the present invention for administration to a human (of approximately 70 kg body weight) is 0.1 mg to 1 g, such as 1 mg to 500 mg of the active ingredient per unit dose, expressed as the weight of free base. The unit dose may be administered, for example, 1 to 4 times per day. It will be appreciated that it may be necessary to make routine variations to the dosage depending on the age and weight of the patient as well as the severity of the condition to be treated. The dosage may also depend on the route of administration. The precise dose and route of administration will ultimately be at the discretion of the attendant physician or veterinarian.

The compounds of formula (I) may also be used in combination with other therapeutic agents. The invention thus provides, in a further aspect, a combination comprising at least one chemical entity chosen from compounds of formula (I) and pharmaceutically acceptable derivative(s) thereof together with one or more further therapeutic agent(s).

When at least one chemical entity chosen from compounds of formula (I) and pharmaceutically acceptable derivative(s) thereof is used in combination with a second therapeutic agent the dose of each compound may differ from that when the compound is used alone. Appropriate doses will be readily appreciated by those skilled in the art. It will be appreciated that the amount of a compound of the invention required for use in treatment will vary with the nature of the condition being treated and the age and the condition of the patient and will be ultimately at the discretion of the attendant physician or veterinarian. The compounds of the present invention may be used in combination with other antithrombotic drugs (such as thrombin inhibitors, thromboxane receptor antagonists, prostacyclin mimetics, phosphodiesterase inhibitors, fibrinogen antagonists, thrombolytic drugs such as tissue plasminogen activator and streptokinase, non-steroidal anti-inflammatory drugs such as aspirin, and the like), anti-hypertensive agents (such as angiotensin-converting enzyme inhibitors, angiotensin-II receptor antagonists, ACE/NEP inhibitors, β-blockers, calcium channel blockers, PDE inhibitors, aldosterone blockers), anti-atherosclerotic/dyslipidaemic agents (such as HMG-CoA reductase inhibitors) and anti-arrhythmic agents.

The combinations referred to above may conveniently be presented for use in the form of a pharmaceutical formulation and thus pharmaceutical formulations comprising a combination as defined above together with at least one pharmaceutically acceptable carrier and/or excipient comprise a further aspect of the invention. The individual components of such combinations may be administered either sequentially or simultaneously in separate or combined pharmaceutical formulations by any convenient route.

When administration is sequential, either the Factor Xa inhibitor or the second therapeutic agent may be administered first. When administration is simultaneous, the combination may be administered either in the same or different pharmaceutical composition.

When combined in the same formulation it will be appreciated that the two compounds must be stable and compatible with each other and the other components of the formulation. When formulated separately they may be provided in any convenient formulation, conveniently in such manner as are known for such compounds in the art.

The chemical entities chosen from compounds of formula (I) and pharmaceutically acceptable derivative(s) thereof may be prepared by the processes described hereinafter, said processes constituting a further aspect of the invention. In the following description, the groups are as defined above for compounds of formula (I) unless otherwise stated.

According to a further aspect of the present invention, there is provided a process (A) for preparing compounds of formula (I) which comprises of reacting compounds of formula (II) or an acid addition salt thereof with compounds of formula (III) where V is a suitable leaving group, such as a halide, e.g. chloride. When the free base of a compound of formula (II) is used, the reaction is conveniently carried out in the presence of a base, e.g. pyridine, and in a suitable solvent, e.g. acetonitrile (MeCN), suitably at 0° C. to room temperature. When the acid addition salt of a compound of formula (II) is used, the reaction is conveniently carried out in the presence of a base, e.g. N,N-diisopropylethylamine (DIPEA), and in a suitable solvent, e.g. MeCN, suitably at 0° C. to room temperature.

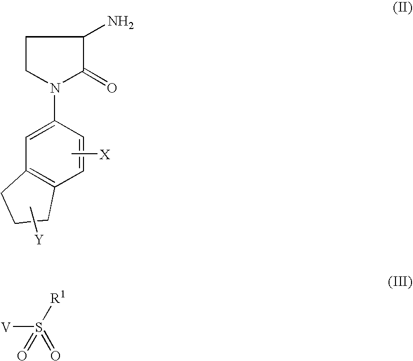

Compounds of formula (I) where R² is a substituent other than hydrogen may be prepared by reacting a compound of formula (I) where R² is hydrogen with a compound of formula (XIII):
R²-T (XIII)
wherein R¹ and R² are defined as above and T is a suitable leaving group such as a halide, e.g. iodide. The reaction is effected in a suitable organic solvent, e.g. THF, DMF, in the presence of a base, e.g. LiHMDS (lithium hexamethyldisilylamide), potassium carbonate or sodium carbonate at a temperature range from −78° C. to +50° C., preferably −78° C. to room temperature. Furthermore, it will appreciated that the substituent R², other than hydrogen, may be introduced at various intermediate stages by methods well known to those skilled in the art.

If X and/or Y contains a group reactive to compounds of formula (III), such groups may be protected prior to reaction of a compound of formula (II) with compounds of formula (III) using methods well known in the art and such protecting groups removed under standard conditions to provide compounds of formula (I) after completion of the reaction of compounds of formula (II) with compounds of formula (III).

Compounds of formula (III) are known compounds or may be prepared by methods known in the literature or processes known to those skilled in the art.

Compounds of formula (II) may be prepared from compounds of formula (IV) by removal of the protecting group P¹, e.g. t-butyloxycarbonyl (Boc), under standard conditions. For example, where P¹ represents Boc, removal of the protecting group may be effected under acidic conditions, using for example hydrogen chloride in a solvent such as dioxane and/or methanol, or trifluoroacetic acid (TFA) in a solvent such as dichloromethane (DCM).

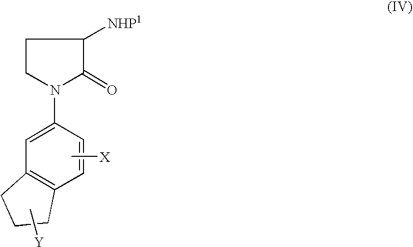

Compounds of formula (IV) may be prepared from compounds of formula (V): 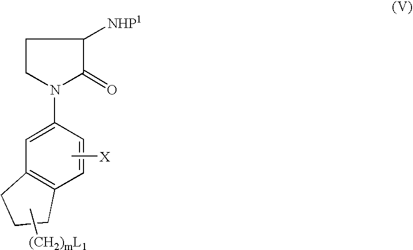
where L₁ is a suitable leaving group such as a hydroxyl group which has been activated, e.g. by reaction with methanesulphonyl chloride in the presence of a suitable base e.g. triethylamine, followed by treatment with HNR^{c}R^{d}, which can be used in excess, in a suitable solvent, e.g. DCM or ethylene glycol dimethylether, suitably at −78° C. to room temperature. Alternatively, a compound of formula (V) where L₁ is a halide e.g. bromide may be prepared from a compound of formula (V) where L₁ is a hydroxyl group by treatment with carbon tetrabromide and triphenylphosphine in a suitable solvent e.g. DCM.

Compounds of formula HNR^{c}R^{d} are known compounds or may be prepared by methods known in the literature or processes known to those skilled in the art.

Compounds of formula (V), where L¹ represents —OH, may be prepared from compounds of formula (VI): 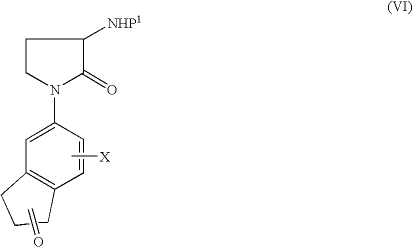
where m=0 by reduction under standard conditions, e.g. by treatment with a nucleophilic hydride source, e.g. sodium borohydride, in a suitable solvent, e.g. methanol, suitably at 0° C. to room temperature;
where m=1 by reaction with an (alkoxymethyl)triphenylphosphonium chloride e.g. (methoxymethyl)triphenylphosphonium chloride and a base, e.g. butyllithium, in a suitable solvent e.g. tetrahydrofuran, suitably at −78° C. to room temperature; followed by treatment with an acid e.g. hydrochloric acid, suitably at room temperature; followed by treatment with a nucleophilic hydride source, e.g. sodium borohydride, in a suitable solvent, e.g. methanol, suitably at 0° C. to room temperature;
where m=2 by reaction with a trialkylphosphonoacetate e.g. trimethylphosphonoacetate and a base e.g. sodium hydride, in a suitable solvent e.g. tetrahydrofuran, suitably at room temperature; followed by hydrogenation suitably at atmospheric pressure with a suitable catalyst e.g 10% palladium on charcoal, in a suitable solvent e.g. ethanol; followed by treatment with a nucleophilic hydride source, e.g. sodium borohydride, in a suitable solvent, e.g. methanol, suitably at 0° C. to room temperature.

Compounds of formula (VI) may be prepared from compounds of formula (VII): 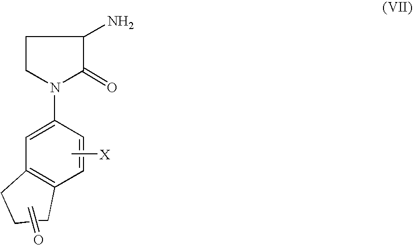
by protection with a suitable P¹ protecting group using methodologies well known to those skilled in the art. See, for example, “Protective groups in organic synthesis” by T. W. Greene and P. G. M. Wuts (John Wiley & sons 1991) or “Protecting Groups” by P. J. Kocienski (Georg Thieme Verlag 1994). For example, where P¹ represents Boc, by treatment with di-tert-butyldicarbonate (bis(1,1-dimethylethyl)dicarbonate, Boc₂O) in a suitable solvent, e.g. DCM, suitably at room temperature.

Compounds of formula (VII) may be prepared by reacting compounds of formula (VIII) with compounds of formula (IX): 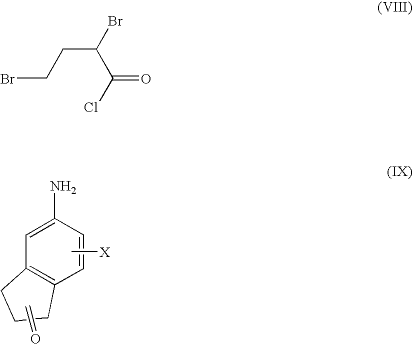
by sequential treatment in a suitable solvent e.g. MeCN, with an acid scavenger e.g. sodium phosphate suitably at −10° C. to +10° C., followed by a base e.g potassium carbonate suitably at −10° C. to +10° C., followed by aqueous ammonia suitably at room temperature to 40° C.

Compounds of formula (VIII) and (IX) are known compounds or may be prepared by methods known in the literature or processes known to those skilled in the art.

Alternatively, compounds of formula (VI) may be prepared from compounds of formula (X): 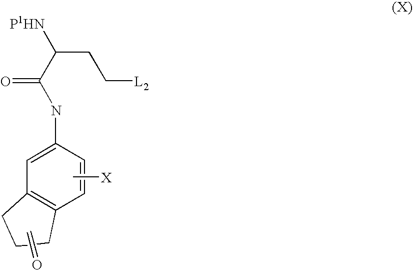
by cyclisation, wherein P¹ is as described above and L² represents a leaving group, e.g. S⁺MeRX⁻. The ring closure may be performed by treatment with Dowex resin in a suitable solvent, e.g. MeCN. Alternatively, the ring closure may be performed by treatment with potassium carbonate in a suitable solvent, e.g. MeCN. Generally R will represent alkyl or aralkyl and X will represent halide, such as iodide.

Compounds of formula (X) may be prepared by reacting compounds of formula (XI) with compounds of formula (IX): 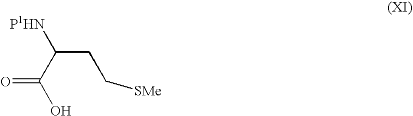
in the presence of a coupling agent, for example 2-(7-azabenzotriazole-1-yl)-1,1,3,3-tetramethyluronium hexafluorophosphate (HATU), followed by treatment with RX, where RX is a compound (e.g. MeI) capable of converting sulphur in the SMe moiety to a sulphonium salt, in a suitable solvent, e.g. acetone.

Compounds of formula (XI) are known compounds or may be prepared by methods known in the literature or processes known to those skilled in the art.

Compounds of formula (I) where one of R^{c} and R^{d} represents hydrogen may be prepared from compounds of formula (XII): 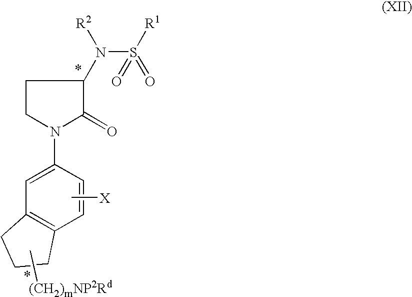
where P² represents a suitable amine protecting group, e.g. trifluoroacetyl, by removal of the protecting group under standard conditions, for example by treatment with potassium carbonate.

Compounds of formula (XII) where R² is a substituent other than hydrogen may be prepared by reacting a compound of formula (XII) where R² is hydrogen with a compound of formula (XIII):
R²—T (XIII)
wherein R¹ and R² are defined as above and T is a suitable leaving group such as a halide, e.g. iodide. The reaction is effected in a suitable organic solvent, e.g. THF, DMF, in the presence of a base, e.g. LiHMDS (lithium hexamethyldisilylamide), potassium carbonate or sodium carbonate at a temperature range from −78° C. to +50° C., preferably −78° C. to room temperature.

Compounds of formula (XII) where R² is hydrogen may be prepared from compounds of formula (XIV): 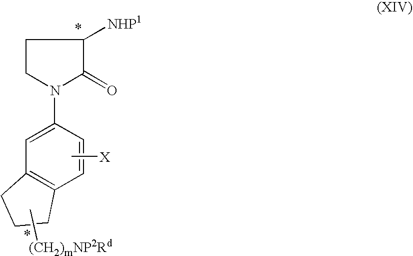
by removal of the protecting group P¹ (as defined above), for example removal of the protecting group may be effected under acidic conditions, using for example hydrogen chloride in a solvent such as dioxane and/or methanol, or trifluoroacetic acid (TFA) in a solvent such as dichloromethane (DCM), followed by reaction with a compound of formula (III).

Compounds of formula (XIV) may be prepared from compounds of formula (IV) where Y represents —(CH₂)ₘNHR^{d} by protection of the amine group under standard conditions, e.g. by treatment with trifluoroacetic anhydride ((CF₃CO)₂O) in a suitable base, e.g. pyridine, and in a suitable solvent, e.g. DCM.

It will be appreciated by those skilled in the art that compounds of formula (I) or a solvate thereof may be synthesized from appropriate intermediates via solid phase chemistry processes.

Those skilled in the art will appreciate that in the preparation of compounds of formula (I) and/or solvates thereof it may be necessary and/or desirable to protect one or more sensitive groups in the molecule or the appropriate intermediate to prevent undesirable side reactions. Suitable protecting groups for use according to the present invention are well known to those skilled in the art and may be used in a conventional manner. See, for example, “Protective groups in organic synthesis” by T. W. Greene and P. G. M. Wuts (John Wiley & sons 1991) or “Protecting Groups” by P. J. Kocienski (Georg Thieme Verlag 1994). Examples of suitable amino protecting groups include acyl type protecting groups (e.g. formyl, trifluoroacetyl, acetyl), aromatic urethane type protecting groups (e.g. benzyloxycarbonyl (Cbz) and substituted Cbz), aliphatic urethane protecting groups (e.g. 9-fluorenylmethoxycarbonyl (Fmoc), t-butyloxycarbonyl (Boc), isopropyloxycarbonyl, cyclohexyloxycarbonyl) and alkyl or aralkyl type protecting groups (e.g. benzyl, trityl, chlorotrityl). Examples of suitable hydroxyl protecting groups may include for example alkyl silyl groups, such as trimethylsilyl or tert-butyldimethylsilyl; alkyl ethers such as tetrahydropyranyl or tert-butyl; or esters such as acetate. Examples of carboxylic acid protecting groups may include for example aralkyl groups, e.g. benzyl, or alkyl groups, e.g. t-butyl.

Various intermediate compounds used in the above-mentioned process, including but not limited to certain compounds of formulae (II), (IV), (V), (XII) and (XIV) constitute a further aspect of the present invention.

The present invention will now be further illustrated by the accompanying examples which should not be construed as limiting the scope of the invention in any way.

All publications, including but not limited to patents and patent applications, cited in this specification are herein incorporated by reference as if each individual publication were specifically and individually indicated to be incorporated by reference herein as though fully set forth.

### EXAMPLES

Abbreviations

MeCN Acetonitrile

DCM Dichloromethane

Boc t-butyloxycarbonyl

HF Tetrahydrofuran

FA Trifluoroacetic acid

HATU 2-(7-Azabenzotriazole-1-yl)-1,1,3,3-tetramethyluronium hexafluorophosphate

m multiplet

MeOH Methanol

q quartet

obs obscured

singlet

t triplet

min minutes

h hours

Intermediate 1

### 3-Amino-1-(1-oxo-2,3-dihydro-1H-inden-5-yl)-2-pyrrolidinone

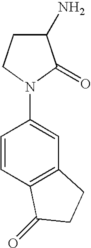

2,4-Dibromobutanoyl chloride (5.16 ml) was added dropwise to a cooled (0° C.) suspension of sodium phosphate (3.06 g) and 5-aminoindan-1-one (5.0 g) in dry MeCN (100 ml). After 2 h at 0° C. potassium carbonate (9.39 g) was added in one portion. The mixture was allowed to warm to room temperature and stirred for 14 h. The suspension was filtered through celite, then aqueous ammonia (0.88 g/ml, 20 ml) was added to the filtrate and the mixture stirred at 40° C. for 14 h, after which all volatiles were removed under reduced pressure. The residue was partitioned between DCM (250 ml) and a mixture of water (200 ml) brine (100 ml) and 2N aqueous sodium hydroxide (1 ml). The aqueous phase was extracted with further DCM and the combined extracts were dried over magnesium sulfate. This DCM solution of the title compound was reduced in volume to ca. 500 ml and taken directly to the next stage.

Mass spectrum: Found: MH⁺ 231

H.p.l.c. Rₜ 1.25 min

Intermediate 2

### 1,1-Dimethylethyl [2-oxo-1-(1-oxo-2,3-dihydro-1H-inden-5-yl)-3-pyrrolidinyl]carbamate

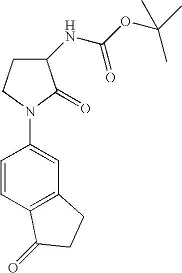

The DCM solution of 3-amino-1-(1-oxo-2,3-dihydro-1H-inden-5-yl)-2-pyrrolidinone (see Intermediate 1) (c. 500 ml) was treated with bis(1,1-dimethylethyl)dicarbonate (8.16 g) and the mixture stirred for 72 h at room temperature. The crude solution was directly loaded onto 4×90 g silica Biotage™ cartridges and eluted with ethyl acetate; and appropriate fractions evaporated to furnish the title compound (6.8 g) as a pale yellow solid.

Mass spectrum: Found: MH⁺ 331

H.p.l.c. Rₜ 2.67 min

Intermediate 3

### 1,1-Dimethylethyl [1-(1-hydroxy-2,3-dihydro-1H-inden-5-yl)-2-oxo-3-pyrrolidinyl]carbamate

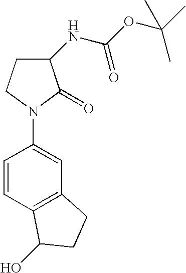

Sodium borohydride (1.56 g) was added portionwise to a stirred solution of 1,1-dimethylethyl [2-oxo-1-(1-oxo-2,3-dihydro-1H-inden-5-yl)-3-pyrrolidinyl]carbamate (see Intermediate 2) (6.8 g) in methanol (500 ml) keeping the temperature below 10° C. The reaction was quenched after 80 min by the addition of saturated aqueous ammonium chloride (100 ml) and all volatiles were removed under reduced pressure. The residue was partitioned between chloroform and water; the organic layer was separated, dried over magnesium sulfate and evaporated to furnish the title compound (6.7 g) as a pale yellow solid.

Mass spectrum: Found: MH⁺ 333

H.p.l.c. Rₜ 2.55 min

Intermediate 4

### 1,1-Dimethylethyl{1-[1-(dimethylamino)-2,3-dihydro-1H-inden-5-yl]-2-oxo-3-pyrrolidinyl}carbamate

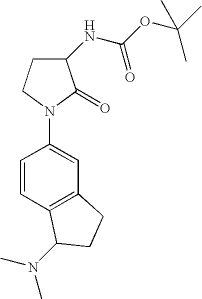

A solution of 1,1-dimethylethyl [1-(1-hydroxy-2,3-dihydro-1H-inden-5-yl)-2-oxo-3-pyrrolidinyl]carbamate (see Intermediate 3) (6.7 g) in DCM (500 ml) was cooled to −78° C. and treated with methanesulfonyl chloride (2.3 ml) and triethylamine (4.2 ml). Stirring was continued for 120 min at −78° C. before allowing the mixture to warm up to room temperature overnight. Dimethylamine (2M solution in THF, 50.4 ml) was added and the mixture was allowed to stir at room temperature for a further 72 h. All volatiles were removed under reduced pressure and the residue was purified using reverse phase liquid chromatography on a CombiFlash® Companion™0 system eluting with a gradient of 10% to 30% MeCN/water containing 0.1% formic acid, and appropriate fractions evaporated to furnish the title compound (3.2 g) as a colourless oil.

Mass spectrum: Found: MH⁺ 360

H.p.l.c. Rₜ 2.02 min

Intermediate 5

### 3-Amino-1-[1-(dimethylamino)-2,3-dihydro-1H-inden-5-yl]-2-pyrrolidinone

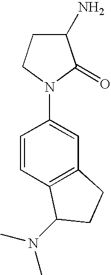

1,1-Dimethylethyl {1-[1-(dimethylamino)-2,3-dihydro-1H-inden-5-yl]-2-oxo-3-pyrrolidinyl}carbamate (see Intermediate 4) (3.2 g) was stirred overnight under nitrogen in methanol (100 ml) and 4N HCl in dioxane (22 ml). All volatiles were removed under reduced pressure and the residue partitioned between saturated aqueous sodium carbonate and ethyl acetate. Six further ethyl acetate extracts were combined and dried over magnesium sulfate, and appropriate fractions evaporated to furnish the free based title compound as a pale yellow oil (1.9 g).

Mass spectrum: Found: MH⁺ 260

H.p.l.c. Rₜ 0.26 min

Intermediate 6

### 1,1-Dimethylethyl((1S)-3-(methylthio)-1-{[(1-oxo-2,3-dihydro-1H-inden-5-yl)amino]carbonyl}propyl)carbamate

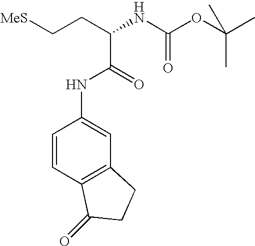

A suspension of N-t-butoxycarbonyl-L-methionine (9.28 g) and HATU (15.0 g) in anhydrous DCM (200 ml) was treated with N,N-diisopropylethylamine (7.14 ml) and stirred for 20 min. The suspension was treated with 5-amino-1-indanone (5.0 g) in 1 g portions over 2 min. The reaction mixture was stirred at room temperature for 20 h and the resulting suspension was partitioned between DCM (2×100 ml) and saturated aqueous sodium hydrogen carbonate solution (150 ml). The organic extracts were combined and washed sequentially with saturated aqueous sodium hydrogen carbonate solution (200 ml) and brine. The organic phase was then passed through a hydrophobic frit and evaporated to afford a black oil. After purification on silica gel (Art 9385) eluting with a DCM:MeOH gradient, the column fractions were combined, evaporated to dryness and re-dissolved in DCM (200 ml) and washed with saturated aqueous sodium hydrogen carbonate solution (3×250 ml), brine, passed through a hydrophobic frit and evaporated to give the title compound as coloured foam (12.55 g, contaminated with (CH₃)₂NCON(CH₃)₂).

Mass spectrum: Found: MH⁺ 379

H.p.l.c. Rₜ 2.97 min

Intermediate 7

### 1,1-Dimethylethyl [(3S)-2-oxo-1-(1-oxo-2,3-dihydro-1H-inden-5-yl)-3-pyrrolidinyl]carbamate

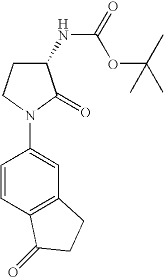

A solution of 1,1-dimethylethyl ((1S)-3-(methylthio)-1-{[(1-oxo-2,3-dihydro-1H-inden-5-yl)amino]carbonyl}propyl)carbamate (see Intermediate 6) (12.5 g) in acetone (100 ml) was treated with iodomethane (20.5 ml) and the solution allowed to stand at room temperature for 21 h then evaporated to dryness to give an orange foam. This foam was dissolved in anhydrous MeCN (40 ml) and treated with caesium carbonate (11.3 g) and stirred at 60° C. (oil bath temperature) for 60 min. The reaction mixture was cooled and filtered through celite washing through with MeCN (250 ml) and DCM (250 ml) and the filtrate evaporated to dryness. The residue was partitioned between water (300 ml) and DCM (1×250 ml and 1×100 ml). The combined organic extracts were combined and washed with brine, passed through a hydrophobic frit and evaporated to afford a solid. Triturating with diethyl ether followed by filtration gave the title compound as a brown solid (7.62 g).

Mass spectrum: Found: MH⁺ 331

H.p.l.c. Rₜ 2.68 min

Intermediate 8

### 1,1-Dimethylethyl[(3S)-1-(1-hydroxy-2,3-dihydro-1H-inden-5-yl)-2-oxo-3-pyrrolidinyl]carbamate

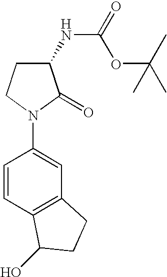

suspension of 1,1-dimethylethyl[(3S)-2-oxo-1-(1-oxo-2,3-dihydro-1H-inden-5-yl)-3-pyrrolidiny]carbamate (see Intermediate 7) (2.0 g) in anhydrous MeOH (50 ml) was stirred under nitrogen and cooled in an ice bath. Sodium borohydride (0.46 g) was added and the reaction mixture was stirred in the cooling bath for 2 h, then evaporated to dryness and partitioned between DCM (150 ml) and water (50 ml). The layers were separated and the aqueous phase washed with DCM (50 ml). The organic extracts were combined, washed with brine, passed through a hydrophobic frit and evaporated to dryness. Purification on a 50 g silica column eluting with [20:1] DCM:MeOH gave the title compound as a yellow solid (1.89 g).

Mass spectrum: Found: MH⁺ 333

H.p.l.c. Rₜ 2.55 min

Intermediate 9

### 1,1-Dimethylethyl{(3S)-1-[1-(dimethylamino)-2,3-dihydro-1H-inden-5-yl]-2-oxo-3-pyrrolidinyl }carbamate

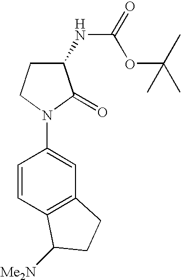

A solution of 1,1-dimethylethyl[(3S)-1-(1-hydroxy-2,3-dihydro-1H-inden-5-yl)-2-oxo-3-pyrrolidinyl]carbamate (see Intermediate 8) (0.98 g) in anhydrous DCM (50 ml) was cooled to −25° C. under nitrogen and the resulting suspension was treated with triethylamine (1.045 ml) and the mixture was then cooled to −76° C. The resulting suspension was treated with a solution of methanesulfonyl chloride (582 ul) in anhydrous DCM (2 ml) which was added over 5 min keeping the temperature below −70° C. The resulting mixture was stirred at −76° C. for 70 min. 2M Dimethylamine in THF (8 ml) was added over 5 min and the cloudy reaction mixture was then stirred for 20 h during which time it slowly reached room temperature. The cloudy reaction mixture was evaporated to dryness and suspended in [95:5] water:MeCN and purified on 2×6 g Oasis® columns eluting with [95:5] water:MeCN and then MeCN. The product fractions were combined and evaporated to give a yellow solid. Purification on a 50 g silica column eluting with [50:1] DCM:MeOH to [5:1] DCM:2N ammonia in MeOH gave the title compound as a colourless waxy solid (0.42 g).

Mass spectrum: Found: MH⁺ 360

H.p.l.c. Rₜ 2.01 min

Intermediate 10

### (3S)-3-Amino-1-[1-(dimethylamino)-2,3-dihydro-1H-inden-5-yl]-2-pyrrolidine

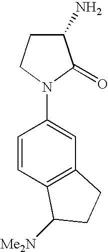

A solution of 1,1-dimethylethyl{(3S)-1-[1-(dimethylamino)-2,3-dihydro-1H-inden-5-yl]-2-oxo-3-pyrrolidinyl}carbamate (see Intermediate 9) (200 mg) in anhydrous DCM (5 ml) and anhydrous MeOH (5 ml) was treated with 4M HCl in dioxane (750 ul) and the resulting solution was stirred at room temperature for 23 h. The reaction mixture was evaporated to dryness and the residue purified on a 20 g SCX column eluting with MeOH to 1N ammonia in MeOH. The product fractions were combined and solvent evaporated to give the title compound as a gum (139 mg).

Mass spectrum: Found: MH⁺ 260

H.p.l.c. Rₜ 0.26 min

Intermediate 11

### 3-Chloro-N-{(3S)-1-[1-(dimethylamino)-2,3-dihydro-1H-inden-5-yl]-2-oxo-3-pyrrolidinyl}-1-[tris(1-methylethyl)silyl]-1H-indole-6-sulfonamide

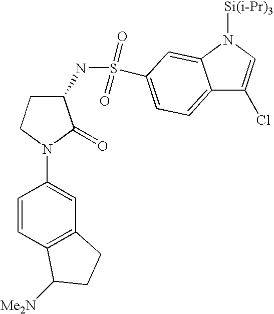

A solution of (3S)-3-amino-1-[1-(dimethylamino)-2,3-dihydro-1H-inden-5-yl]-2-pyrrolidine (see Intermediate 10) (46.4 mg) in anhydrous MeCN (3 ml) was stirred under nitrogen, cooled in an ice bath and treated with pyridine (43.5 ul). The reaction mixture was then treated with a solution of 3-chloro-1-[tris(1-methylethyl)silyl]-1H-indole-6-sulfonyl chloride (see Intermediate 26) (94.6 mg) in anhydrous MeCN (5 ml) over 10 min. The reaction mixture was stirred in the cooling bath for 20 min giving a yellow suspension. The cooling bath was removed and the suspension was stirred at room temperature for 2 h and stood at room temperature for 4 days. The suspension was then evaporated to dryness and partitioned between saturated aqueous sodium hydrogen carbonate solution (10 ml) and DCM (30 ml). The layers were separated and the aqueous layer washed with DCM (20 ml). The organic extracts were combined, washed with brine, passed through a hydrophobic frit and evaporated to an orange gum which was purified on a 20 g silica SPE column eluting with [25:1] to [10:1] DCM:MeOH. The product fractions were combined and evaporated to afford the title compound as a colourless foam (60.9 mg).

Mass spectrum: Found: MH⁺ 629

H.p.l.c. Rₜ 3.21 min

Also isolated was 3-chloro-N-{(3S)-1-[1-(dimethylamino)-2,3-dihydro-1H-inden-5-yl]-2-oxo-3-pyrrolidinyl}-1H-indole-6-sulfonamide (Example 5) (11.1 mg).

Mass spectrum: Found: MH⁺ 473

H.p.l.c. Rₜ 2.43 min

Intermediate 12

### 1,1-Dimethylethyl{(3S)-1-[1-(methylamino)-2,3-dihydro-1H-inden-5-yl]-2-oxo-3-pyrrolidinyl}carbamate

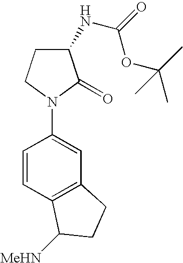

A solution of 1,1-dimethylethyl[(3S)-1-(1-hydroxy-2,3-dihydro-1H-inden-5-yl)-2-oxo-3-pyrrolidinyl]carbamate (see Intermediate 8) (1.42 g) in anhydrous DCM (150 ml) was cooled to −30° C. under nitrogen and the resulting suspension was treated with triethylamine (1.5 ml) and the mixture was then cooled to −76° C. The resulting suspension was treated with a solution of methanesulphonyl chloride (832 ul) in anhydrous DCM (20 ml) over 10 min keeping the temperature below −75° C. The resulting mixture was stirred at −76° C. for 70 min. 2M Methylamine in THF (90 ml) was added over 15 min and the suspension was stirred for 20 h during which time it slowly reached room temperature. The suspension was evaporated to dryness, and purification on a 70 g SCX column eluting with MeOH to 1N ammonia in MeOH gave the title compound as a yellow solid (1.25 g).

Mass spectrum: Found: MH⁺ 315 (eliminates to the indene in the ms)

H.p.l.c. Rₜ 2.04 min

Intermediate 13

### 1,1-Dimethylethyl((3S)-1-{1-[methyl(trifluoroacetyl)amino]-2,3-dihydro-1H-inden-5-yl}-2-oxo-3-pyrrolidinyl)carbamate

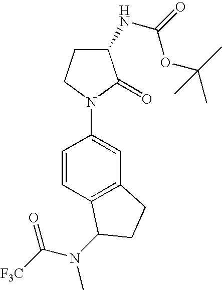

A solution of 1,1-dimethylethyl{(3S)-1-[1-(methylamino)-2,3-dihydro-1H-inden-5-yl]-2-oxo-3-pyrrolidinyl}carbamate (see Intermediate 12) (570 mg) in anhydrous DCM (30 ml) was cooled to 0° C. under nitrogen and treated with pyridine (267 ul) followed by a solution of trifluoroacetic anhydride (326 ul) in anhydrous DCM (2.5 ml) over 5 min and stirred at 0° C. for 30 min. The mixture was diluted with DCM (20 ml) and washed with water (25 ml), 1N HCl (40 ml), brine (25 ml), passed through a hydrophobic frit and evaporated to dryness.

The yellow solid was purified on a 50 g silica SPE column eluting with a DCM:MeOH gradient to give the title compound as a cream solid (480 mg).

Mass spectrum: Found: MH⁺ 442

H.p.l.c. Rₜ 3.20 min

Intermediate 14

### N-{5-[(3S)-3-Amino-2-oxo-1-pyrrolidinyl]-2,3-dihydro-1H-inden-1-yl}-2,2,2-trifluoro-N-methylacetamide hydrochloride

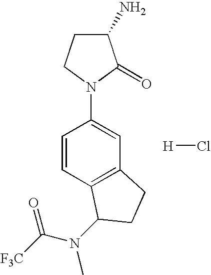

A solution of 1,1-dimethylethyl ((3S)-1-{1-[methyl(trifluoroacetyl)amino]-2,3-dihydro-1H-inden-5-yl}-2-oxo-3-pyrrolidinyl)carbamate (see Intermediate 13) (478 mg) in anhydrous DCM (10 ml) and anhydrous MeOH (10 ml) was treated with 4M HCl in dioxane (1.8 ml) and stirred at room temperature for 24 h. The reaction mixture was evaporated to dryness to give the title compound as a cream solid (415 mg, >100%).

Mass spectrum: Found: MH⁺ 342

H.p.l.c. Rₜ 2.13 min

Intermediate 15

### N-[5-((3S)-3-{[(6-Chloro-2-naphthalenyl)sulfonyl]amino}-2-oxo-1-pyrrolidinyl)-2,3-dihydro-1H-inden-1-yl]-2,2,2-trifluoro-N-methylacetamide

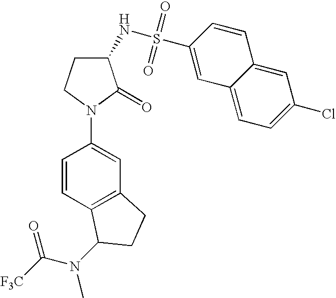

A light suspension of N-{5-[(3S)-3-amino-2-oxo-1-pyrrolidinyl]-2,3-dihydro-1H-inden-1-yl}-2,2,2-trifluoro-N-methylacetamide hydrochloride (see Intermediate 14) (310 mg) in anhydrous MeCN (20 ml) was cooled in an ice bath and treated with N,N-diisopropylethyl amine (150 ul) and pyridine (200 ul). A solution of 6-chloro-2-naphthalenesulfonyl chloride (257 mg) in anhydrous MeCN (5 ml) was added over 5 min and the yellow mixture was stirred in the ice bath for 30 min. The cooling bath was removed and reaction mixture was stirred at room temperature for 3 h, evaporated to dryness and partitioned between water (40 ml) and DCM (50 ml). The layers were separated and the aqueous layer washed with DCM (40 ml). The organic extracts were combined washed with saturated aqueous sodium hydrogen carbonate solution (50 ml), brine, passed through a hydrophobic frit and evaporated to a yellow gum. Repeated purification on a 50 g silica SPE column eluting with [100:1] DCM: MeOH gave the title compound as a pale yellow foam (206 mg).

Mass spectrum: Found: MH⁺ 566

H.p.l.c. Rₜ 3.62 min

Intermediate 16

### N-{5-[(3S)-3-({[(E)-2-(5-Chloro-2-thienyl)ethenyl]sulfonyl}amino)-2-oxo-1-pyrrolidinyl]-2,3-dihydro-1H-inden-1-yl }-2,2,2-trifluoro-N-methylacetamide

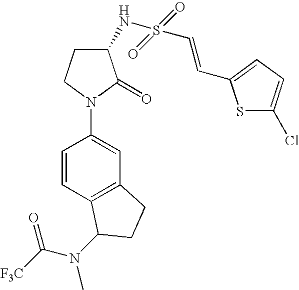

The title compound was prepared from N-{5-[(3S)-3-amino-2-oxo-1-pyrrolidinyl]-2,3-dihydro-1H-inden-1-yl}-2,2,2-trifluoro-N-methylacetamide hydrochloride (see Intermediate 14) and (E)-2-(5-chloro-2-thienyl)ethenesulfonyl chloride using the procedure described for Intermediate 15.

Mass spectrum: Found: MH⁺ 548

H.p.l.c. Rₜ 3.50 min

Intermediate 17

### N-{5-[(3S)-3-({[2-(5-Chloro-2-thienyl)ethyl]sulfonyl}amino)-2-oxo-1-pyrrolidinyl]-2,3-dihydro-1H-inden-1-yl }-2,2,2-trifluoro-N-methylacetamide

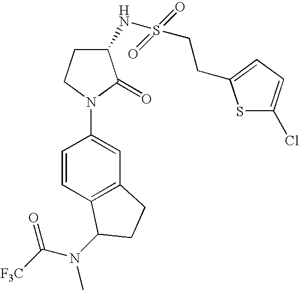

The title compound was prepared from N-{5-[(3S)-3-amino-2-oxo-1-pyrrolidinyl]-2,3-dihydro-1H-inden-1-yl}-2,2,2-trifluoro-N-methylacetamide hydrochloride (see Intermediate 14) and 2-(5-chloro-2-thienyl)ethanesulfonyl chloride using the procedure described for Intermediate 15.

Mass spectrum: Found: MH⁺ 550

H.p.l.c. Rₜ 3.54 min

Intermediate 18

### N-(5-{(3S)-3-[({3-Chloro-1-[tris(1-methylethyl)sily]-1H-indol-6-yl}sulfonyl)amino]-2-oxo-1-pyrrolidinyl}-2,3-dihydro-1H-inden-1-yl)-2,2,2-trifluoro-N-methylacetamide

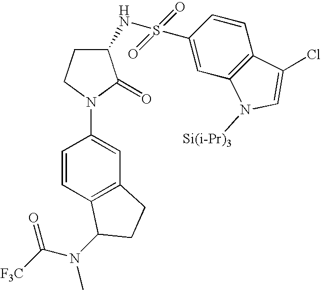

The title compound (containing 30% desilylated material) was prepared from N-{5-[(3S)-3-amino-2-oxo-1-pyrrolidinyl]-2,3-dihydro-1H-inden-1-yl}-2,2,2-trifluoro-N-methylacetamide hydrochloride (see Intermediate 14) and 3-chloro-1-[tris(1-methylethyl)silyl]-1H-indole-6-sulfonyl chloride (see Intermediate 26) using the procedure described for Intermediate 15.

Mass spectrum: Found: MH⁺ 711 and 555 (desilylated material)

H.p.l.c. Rₜ 4.24 min and 3.44 min (desilylated material)

Intermediate 19

### N-[5-((3S)-3-{[(3-Chloro-1H-indol-6-yl)sulfonyl]amino}-2-oxo-1-pyrrolidinyl)-2,3-dihydro-1H -inden-1-yl]-2,2,2-trifluoro-N-methylacetamide

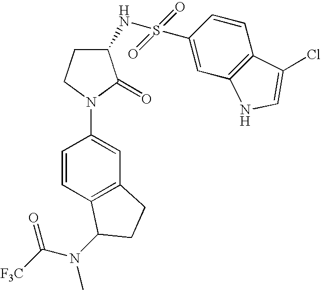

A solution of N-(5-{(3S)-3-[({3-chloro-1-[tris(1-methylethyl)silyl]-1H-indol-6-yl}sulfonyl)amino]-2-oxo-1-pyrrolidinyl}-2,3-dihydro-1H-inden-1-yl)-2,2,2-trifluoro-N-methylacetamide (Intermediate 18) (175 mg) in anhydrous THF (50 ml) and glacial acetic acid (1 ml) was treated with tetraethylammonium fluoride hydrate (55.0 mg) in one portion. The pale yellow solution was stirred at room temperature for 1 h, treated with saturated aqueous ammonium chloride solution (3 ml) and water (3 ml) and evaporated to near dryness. The gummy residue was partitioned between water (10 ml) and DCM (2×25 ml). The organic extracts were combined, washed with brine, passed through a hydrophobic frit and evaporated to dryness. Purification on a 20 g silica SPE column eluting with [50:1] to [30:1] DCM:MeOH gave the title compound as a cream solid (141.0 mg).

Mass spectrum: Found: MH⁺ 555

H.p.l.c. Rₜ 3.43 min

Intermediate 20

### 1,1-Dimethylethyl[(3S)-1-(1-amino-2,3-dihydro-1H-inden-5-yl)-2-oxo-3-pyrrolidinyl]carbamate

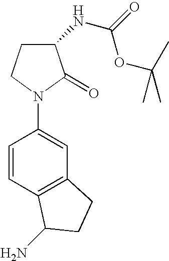

A solution of 1,1-dimethylethyl [(3S)-1-(1-hydroxy-2,3-dihydro-1H-inden-5-yl)-2-oxo-3-pyrrolidinyl]carbamate (see Intermediate 8) (1.0 g) in anhydrous DCM (100 ml) was cooled to −76° C. under nitrogen and the resulting gel was treated with triethylamine (1.05 ml) followed by a solution of methanesulphonyl chloride (0.58 ml) in anhydrous DCM (2 ml) which was added over 5 min keeping the temperature below −70° C. The resulting mixture was stirred at −76° C. for 135 min. 0.5N Ammonia in dioxane (100 ml) was added over 15 min keeping the temperature below −60° C. and the thick reaction mixture was treated with anhydrous DCM (100 ml) and stirred for 20 h during which time it slowly reached room temperature. The cloudy reaction mixture was treated with 0.5N ammonia in dioxane (50 ml) and stirred at room temperature for 1 h, evaporated to dryness and purified on 70 g SCX column eluting with MeOH to 2N ammonia in MeOH. The product fractions were combined and evaporated to give the title compound as a yellow solid (0.53 g).

Mass spectrum: Found: MH⁺ 315 (eliminates to the indene in the ms)

H.p.l.c. Rₜ 2.02 min

Intermediate 21

### 1,1-Dimethylethyl((3S)-2-oxo-1-{1-[(trifluoroacetyl)amino]-2,3-dihydro-1H-inden-5-yl}-3-pyrrolidinyl)carbamate

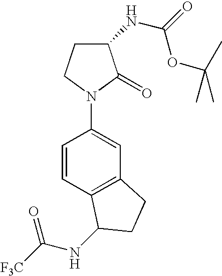

A solution of 1,1-dimethylethyl [(3S)-1-(1-amino-2,3-dihydro-1H-inden-5-yl)-2-oxo-3-pyrrolidinyl]carbamate (Intermediate 20) (300 mg) in anhydrous DCM (15 ml) was cooled to 0° C. under nitrogen and treated with pyridine (146.4 ul) followed by a solution of trifluoroacetic anhydride (166.2 ul) and stirred at 0° C. for 30 min. The cooling bath was removed and the reaction mixture was stirred for 1 h. The mixture was diluted with DCM (5 ml) and treated with a further portion of trifluoroacetic anhydride (166.2 ul). The suspension was stirred for 1 h, diluted with DCM (125 ml) and washed with water (25 ml), brine (25 ml), passed through a hydrophobic frit and evaporated to dryness. The crude product was purified on a 50 g silica SPE column eluting with [25:1] DCM:MeOH to give the title compound as a cream solid (341 mg).

Mass spectrum: Found: MH⁺ 428

H.p.l.c. Rₜ 3.04 min

Intermediate 22

### N-{5-[(3S)-3-Amino-2-oxo-1-pyrrolidinyl]-2,3-dihydro-1H-inden-1-yl}-2,2,2-trifluoroacetamide hydrochloride

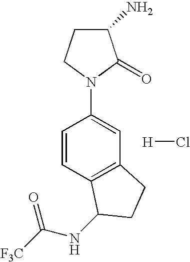

A solution of 1,1-dimethylethyl ((3S)-2-oxo-1-{1-[(trifluoroacetyl)amino]-2,3-dihydro-1H-inden-5-yl}-3-pyrrolidinyl)carbamate (see Intermediate 21) (331 mg) in anhydrous DCM (10 ml) and anhydrous MeOH (15 ml) was treated with 4M HCl in dioxane (1.0 ml) and stirred at room temperature for 4 days. The orange solution was evaporated to dryness to give the title compound as a light brown solid (297 mg, >100%).

Mass spectrum: Found: MH⁺ 328

H.p.l.c. Rₜ 1.86 min

Intermediate 23

### N-[5-((3S)-3-{[(6-Chloro-2-naphthalenyl)sulfonyl]amino}-2-oxo-1-pyrrolidinyl)-2,3-dihydro-1H -inden-1-yl]-2,2,2-trifluoroacetamide

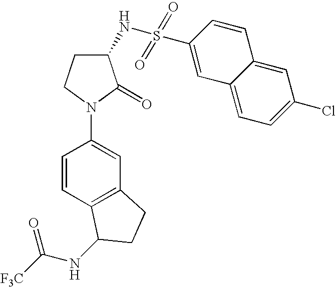

A suspension of N-{5-[(3S)-3-amino-2-oxo-1-pyrrolidinyl]-2,3-dihydro-1H-inden-1-yl}-2,2,2-trifluoroacetamide hydrochloride (Intermediate 22) (145 mg) in anhydrous MeCN (20 ml) was cooled in an ice bath and treated with N,N-diisopropylethyl amine (69.4 ul) and pyridine (96.8 ul). 6-Chloro-2-naphthalenesulfonyl chloride (125 mg) was added in a single portion and the suspension was stirred in the ice bath for 30 min. The cooling bath was removed and reaction mixture was stirred at room temperature for 1 h, diluted with anhydrous MeCN (10 ml) and stirred at room temperature for 18 h. The thick suspension was evaporated to dryness and partitioned between DCM (2×50 ml) and saturated aqueous sodium hydrogen carbonate solution (30 ml). The organic extracts were combined, washed with brine, passed through a hydrophobic frit and evaporated to a yellow solid. Purification on 20 g silica SPE column eluting with [50:1] DCM:MeOH gave a sticky orange solid. This solid was triturated with diethyl ether and the title compound collected by filtration (155 mg).

Mass spectrum: Found: MH⁺ 552

H.p.l.c. Rₜ 3.51 min

Intermediate 24

### N-(5-{(3S)-3-[[(6-Chloro-2-naphthalenyl)sulfonyl](methyl)amino]-2-oxo-1-pyrrolidinyl}-2,3-dihydro-1H-inden-1-yl)-2,2,2-trifluoro-N-methylacetamide

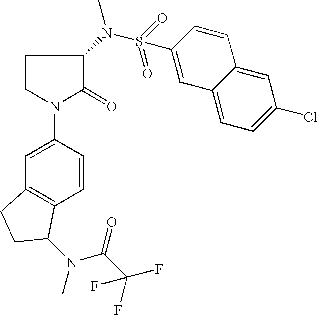

A solution of N-[5-((3S)-3-{[(6-chloro-2-naphthalenyl)sulfonyl]amino}-2-oxo-1-pyrrolidinyl)-2,3-dihydro-1H-inden-1-yl]-2,2,2-trifluoro-N-methylacetamide (see Intermediate 15) (140 mg) in anhydrous DMF (5 ml) was stirred under nitrogen, cooled in an ice bath and treated with potassium carbonate (41 mg) followed by iodomethane (18.6 ul). The cooling bath was removed and the reaction was stirred at room temperature for 2 h then treated further with potassium carbonate (41 mg) and iodomethane (18.6 ul), stirring for an additional 1 h at room temperature. The reaction mixture was then evaporated to dryness and partitioned between water (20 ml) and ethyl acetate (20 ml). The layers were separated and the aqueous layer washed with ethyl acetate (20 ml). The organic extracts were combined, passed through a hydrophobic frit and evaporated to afford the title compound as a white foam (113 mg).

Mass spectrum: Found: MNH₄⁺ 597

H.p.l.c. Rₜ 3.76 min

Intermediate 25:

### 6-Bromo-1-[tris(1-methylethyl)silyl]-1H-indole

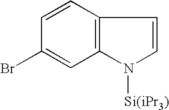

A solution of 6-bromo-1H-indole (2.0 g) in dry THF (20 mL) at 0° C. was treated with sodium hydride (60% dispersion in mineral oil; 0.48 g), in portions, stirring for 30 min. Chloro[tris(1-methylethyl)]silane was added to the reaction in a drop-wise manner, allowing the reaction to warm up to ambient temperature and stirring for 18 h. The reaction was concentrated under reduced pressure, partitioning the residue between DCM and saturated aqueous sodium bicarbonate solution. The separated organic phase was passed through a hydrophobic frit and re-concentrated to a small volume before being loaded onto a pre-conditioned silica phase SPE column (150 mL/70 g) eluting with cyclohexane:ethyl acetate (0-2%). Fractions containing product were combined and concentrated under reduced pressure to give the title compound (3.51 g) as colourless oil.

Mass spectrum: Found: MH⁺ 352/354

H.p.l.c. Rₜ 4.51 min

Intermediate 26:

### 3-Chloro-1-[tris(1-methylethyl)silyl]-1H-indole-6-sulfonyl chloride

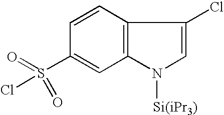

A solution of 6-bromo-1-[tris(1-methylethyl)silyl]-1H-indole (Intermediate 25) (2.30 g) in dry THF (45 mL) at −78° C. was treated with n-butyllithium (1.6M in hexanes; 4.37 mL) in a drop-wise manner. The mixture was allowed to stir for 1 h and then poured into a stirred solution of sulphuryl chloride (1.22 mL in dry cyclohexane (45 mL)) at 0° C. The reaction was allowed to warm up to ambient temperature and stirred for 2.5 h, then quenched with water (25 mL) stirring for 30 min. The separated organic layer was passed through a hydrophobic frit and concentrated under reduced pressure. The crude material was dissolved in cyclohexane and loaded onto a pre-conditioned silica phase SPE column (150 mL/70 g) eluting with cyclohexane:ethyl acetate (0-10%). Fractions containing product were combined and concentrated under reduced pressure to give the title compound (1.18 g) as a beige gum.

Following dimethylamine quench:

Mass spectrum: Found: MH⁺ 415

H.p.l.c. Rₜ 4.22 min

Also identified was the deprotected product 3-chloro-1H-indole-6-sulfonyl chloride.

Following dimethylamine quench:

Mass spectrum: Found: MH⁺ 259

H.p.l.c. Rₜ 3.03 min

### Example 1

### 6-Chloro-N-{1-[1-(dimethylamino)-2,3-dihydro-1H-inden-5-yl]-2-oxo-3-pyrrolidinyl}-2-naphthalenesulfonamide hydrochloride

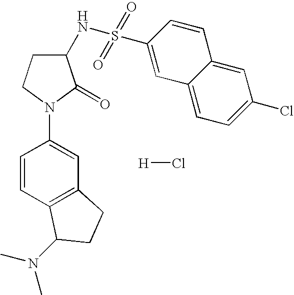

3-Amino-1-[1-(dimethylamino)-2,3-dihydro-1H-inden-5-yl]-2-pyrrolidinone (see Intermediate 5) (0.95 g) was dissolved in dry MeCN (100 ml) and cooled to 0° C. under nitrogen. Pyridine (0.89 ml) was added followed by 6-chloro-2-naphthalenesulfonyl chloride (1.15 g) and the solution allowed to stir overnight at room temperature. The yellow precipitate was filtered off, washed with MeCN and dried in vacuo to afford the title compound.

Mass spectrum: Found: MH⁺ 484

H.p.l.c. Rₜ 2.56 min

### Example 2

### (E)-2-(5-Chloro-2-thienyl)-N-{1-[1-(dimethylamino)-2,3-dihydro-1H-inden-5-yl]-2-oxo-3-pyrrolidinyl}ethenesulfonamide hydrochloride

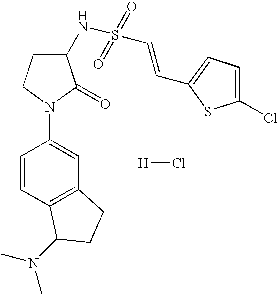

The title compound was prepared from 3-amino-1-[1-(dimethylamino)-2,3-dihydro-1H-inden-5-yl]-2-pyrrolidinone (see Intermediate 5) and (E)-2-(5-chloro-2-thienyl)ethenesulfonyl chloride using the procedure described for Example 1.

Mass spectrum: Found: MH⁺ 466

H.p.l.c. Rₜ 2.40 min

### Example 3

### 6-Chloro-N-{1-[1-(dimethylamino)-2,3-dihydro-1H-inden-5-yl]-2-oxo-3-pyrrolidinyl}-1-benzothiophene-2-sulfonamide

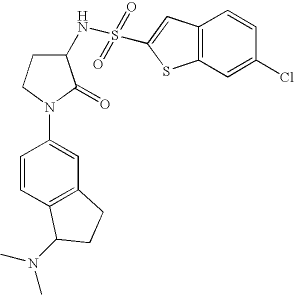

The title compound was prepared from 3-Amino-1-[1-(dimethylamino)-2,3-dihydro-1H-inden-5-yl]-2-pyrrolidinone (see Intermediate 5) and 6-chloro-1-benzothiophene-2-sulfonyl chloride using the procedure described for Example 1 followed by purification on an SCX SPE column eluting with methanol then [1:9] 2N aqueous ammonia: methanol.

Mass spectrum: Found: MH⁺ 490

H.p.l.c. Rₜ 2.58 min

### Example 4

### 6-Chloro-N-{(3S)-1-[-(dimethylamino)-2,3-dihydro-1H-inden-5-yl]-2-oxo-3-pyrrolidinyl-2-naphthalenesulfonamide

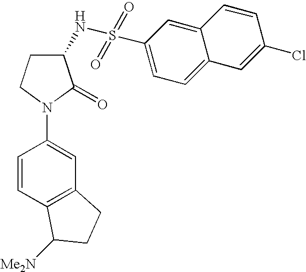

A solution of (3S)-3-amino-1-[1-(dimethylamino)-2,3-dihydro-1H-inden-5-yl]-2-pyrrolidine (see Intermediate 10) (150 mg) in anhydrous MeCN (10 ml) was stirred under nitrogen, cooled to 0° C. and treated with pyridine (140.3 ul). The reaction mixture was then treated with solid 6-chloro-naphthalenesulfonyl chloride (182 mg) in 3 portions. The reaction mixture was stirred at 0° C. for 30 min giving a yellow suspension. The cooling bath was removed and the mixture was stirred at room temperature for 19 h, partitioned between saturated aqueous sodium hydrogen carbonate solution (30 ml) and DCM (40 ml). The layers were separated and the aqueous layer washed with DCM (20 ml). The organic extracts were combined, washed with brine, passed through a hydrophobic frit and evaporated to an orange gum which was purified on a 20 g silica SPE column eluting with [20:1] to [10:1] DCM:MeOH. The product fractions were combined and evaporated to give a colourless foam. This foam was treated with diethyl ether and the colourless title compound was collected by filtration (151 mg).

Mass spectrum: Found: MH⁺ 484

H.p.l.c. Rₜ 2.55 min

¹H nmr (CDCl₃) δ: 2.06 (2H, m), 2.19 (1H, m), 2.22 (6H, s), 2.68-2.97 (3H, m), 3.77 (2H, m), 3.90 (1H, m), 4.29 (1H, m), 5.51 (1H, brs), 7.28 (1H, m), 7.33 (1H, m), 7.44 (1H, brs), 7.56 (1H, m), 7.93 (4H, m), 8.49 (1H, s)

100 mg of 6-chloro-N-{(3S)-1-[-(dimethylamino)-2,3-dihydro-1H-inden-5-yl]-2-oxo-3-pyrrolidinyl-2-naphthalenesulfonamide was separated by chiral preparative chromatography (Chiralpak AD 35% ethanol/heptane (0.1% TFA)) to give Isomer A (TFA salt) as a cream solid (34.7 mg) Rₜ 25.04 min

¹H nmr (CDCl₃) δ: 2.06 (2H, m), 2.18 (1H, m), 2.22 (6H, s), 2.69-2.97 (3H, m), 3.77 (2H, m), 3.90 (1H, m), 4.30 (1H, m), 5.49 (1H, brs), 7.28 (1H, m), 7.33 (1H, m), 7.44 (1H, brs), 7.56 (1H, m), 7.93 (4H, m), 8.49 (1H, s)

Isomer B (TFA salt) as a cream solid (32.6 mg) Rₜ 30.14 min

¹H nmr (CDCl₃) δ: 2.06 (2H, m), 2.18 (1H, m), 2.23 (6H, s, N(CH₃)₂), 2.69-2.97 (3H, m), 3.77 (2H, m), 3.90 (1H, m), 4.30 (1H, m), 5.50 (1H, brs), 7.28 (1H, m), 7.33 (1H, m), 7.44 (1H, brs), 7.56 (1H, m), 7.93 (4H, m), 8.49 (1H, s)

### Example 5

### 3-Chloro-N-{(3S)-1-[1-(dimethylamino)-2,3-dihydro-1H-inden-5-yl]-2-oxo-3-pyrrolidinyl}-1H-indole-6-sulfonamide

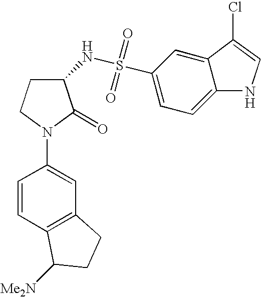

A solution of 3-chloro-N-{(3S)-1-[1-(dimethylamino)-2,3-dihydro-1H-inden-5-yl]-2-oxo-3-pyrrolidinyl}-1-[tris(1-methylethyl)silyl]-1H-indole-6-sulfonamide (see Intermediate 11) (58.0 mg) in anhydrous THF (20 ml) was treated with tetraethylammonium fluoride hydrate (20.6 mg) and the solution was stirred at room temperature for 90 min, quenched with saturated aqueous ammonium chloride solution (2 ml), stirred for 30 min and evaporated to dryness. The residue was suspended in MeOH and purified on a 20 g SCX column eluting with MeOH to 1N ammonia in MeOH. The product fractions were combined and evaporated to a cream solid and combined with 11.1 mg from a previous reaction and purified on a 10 g silica SPE column eluting with [33:1] to [10:1] DCM:2N ammonia in MeOH. The product fractions were evaporated to dryness and the residue was evaporated from DCM and diethyl ether to give the title compound as a cream solid (44.2 mg).

Mass spectrum: Found: MH⁺ 473

H.p.l.c. Rₜ 2.43 min

¹Hnmr (DMSO-d₆) δ: 1.65 (1H, m), 1.95 (3H, m), 2.10 (6H, s, N(CH₃)₂), 2.69-2.87 (2H, m), 3.61 (2H, m), 4.22 (2H, m), 7.22 (1H, d), 7.42 (1H, m), 7.63 (2H, m), 7.82 (1H, d), 7.98 (1H, d), 8.17 (1H, d), 11.91 (1H, brs).

### Example 6

### 2-(5-Chloro-2-thienyl)-N-{(3S)-1-[1-(dimethylamino)-2,3-dihydro-1H-inden-5-yl]-2-oxo-3-pyrrolidinyl }ethanesulfonamide

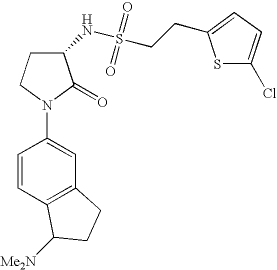

The title compound was prepared from (3S)-3-amino-1-[1-(dimethylamino)-2,3-dihydro-1H -inden-5-yl]-2-pyrrolidine (see Intermediate 10) and 2-(5-chloro-2-thienyl)ethanesulfonyl chloride using the procedure described for Example 1.

Mass spectrum: Found: MH⁺ 468

H.p.l.c. Rₜ 2.48 min

¹Hnmr δ (CDCl₃) 2.09 (3H, m), 2.25 (6H, s), 2.72-3.0 (3H, m), 3.27-3.58 (4H, m), 3.82 (2H, m), 4.33 (2H, m), 4.96 (1H, brs), 6.69 (1H, d), 6.73 (1H, d), 7.37 (2H, m), 7.51 (1H, d)

### Example 7

### 6-Chloro-N-{(3S)-1-[1-(methylamino)-2,3-dihydro-1H-inden-5-yl]-2-oxo-3-pyrrolidinyl}-2-naphthalenesulfonamide

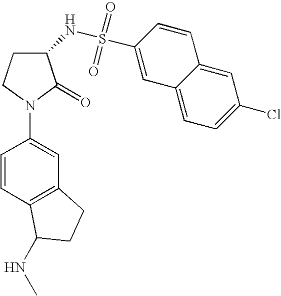

N-[5-((3S)-3-{[(6-chloro-2-naphthalenyl)sulfonyl]amino}-2-oxo-1-pyrrolidinyl)-2,3-dihydro-1H-inden-1-yl]-2,2,2-trifluoro-N-methylacetamide (see Intermediate 15) (200 mg) in MeOH (5 ml) was treated with a solution of sodium carbonate (374.5 mg) in water (1.75 ml). As a thick precipitate formed, MeCN (5 ml) was added and the still thick suspension was heated at 40° for 1 h. Water (1.75 ml) was added and the reaction mixture was stirred at 40° C. for 19 h. The reaction mixture was cooled and evaporated to remove volatiles. The residue was partitioned between water (20 ml) and ethyl acetate (75 ml). A sticky precipitate formed which was collected. The layers in the filtrate were separated and the aqueous phase washed with DCM and [95:5] DCM:MeOH. The organic extracts were combined with the solid residue and evaporated to dryness and purified on a 20 g SCX column eluting with MeOH to 1N ammonia in MeOH and the product fractions were combined and evaporated to dryness. The residue was dissolved in DCM (1 ml) and diluted slowly with diethyl ether. The title compound was collected by filtration as a cream solid (70.0 mg).

Mass spectrum: Found: MH⁺ 468

H.p.l.c. Rₜ 2.62 min

¹Hnmr (CDCl₃) δ: 1.84 (1H, m), 1.90-2.8 (very broad peak, obs), 2.18 (1H, m), 2.40 (1H, m) 2.48 (3H, d), 2.69-2.85 (2H, m), 2.97 (1H, m), 3.74 (2H, m), 3.90 (1H, m), 4.13 1H, t), 7.25-7.32 (m, obs), 7.43 (1H, m), 7.57 (1H, m), 7.90-7.97 (4H, m), 8.49 (1H, s)

### Example 8

### (E)-2-(5-Chloro-2-thienyl)-N-{(3S)-1-[1-(methylamino)-2,3-dihydro-1H-inden-5-yl]-2-oxo-3-pyrrolidinyl}ethenesulfonamide

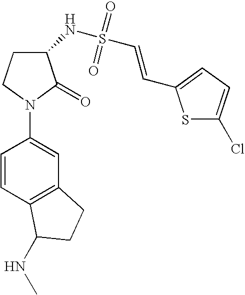

A solution of N-{5-[(3S)-3-({[(E)-2-(5-chloro-2-thienyl)ethenyl]sulfonyl}amino)-2-oxo-1-pyrrolidinyl]-2,3-dihydro-1H-inden-1-yl}-2,2,2-trifluoro-N-methylacetamide (see Intermediate 16) (138 mg) in 2N ammonia in MeOH (30 ml) was stirred at room temperature for 19 h and evaporated to dryness. Purification on a 20 g SCX column eluting with MeOH to 0.5N ammonia in MeOH followed by purification on a 20 g silica SPE column eluting with [20:1] to [10:1] DCM:2N ammonia in MeOH and trituration with diethyl ether followed by filtration gave the title compound as a pale pink solid (67.9 mg).

Mass spectrum: Found: MH⁺ 450

H.p.l.c. Rₜ 2.44 min

¹H nmr (CDCl₃) δ: 1.88 (1H, m), 2.0-3.0 (very broad peak), 2.18 (1H, m), 2.43 (1H, m), 2.50 (3H, d), 2.78 (2H, m), 3.02 (1H, m), 3.78 (2H, m), 4.09 (1H, m), 4.16 (1H, t), 6.59 (1H, d), 6.69 (1H, d), 7.08 (1H, d), 7.29-7.39 (2H, m), 7.45-7.54 (2H, m)

### Example 9

### 2-(5-Chloro-2-thienyl)-N-{(3S)-1-[1-(methylamino)-2,3-dihydro-1H-inden-5-yl]-2-oxo-3-pyrrolidinyl}ethanesulfonamide

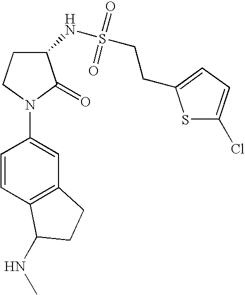

The title compound was prepared from N-{5-[(3S)-3-({[2-(5-chloro-2-thienyl)ethyl]sulfonyl}amino)-2-oxo-1-pyrrolidinyl]-2,3-dihydro-1H-inden-1-yl}-2,2,2-trifluoro-N-methylacetamide (see Intermediate 17) using the procedure described for Example 8.

Mass spectrum: Found: MH⁺ 452

H.p.l.c. Rₜ 2.45 min

¹H nmr (CDCl₃) δ: 1.87 (1H, m), 2.06 (1H, m), 2.2-3.0 (very broad peak, obs) 2.43 (1H, m), 2.51 (3H, d), 2.74 (1H, m), 2.85 (1H, m), 3.04 (1H, m), 2.26-3.58 (4H, m), 3.78 (2H, m), 4.16 (1H, t), 4.32 (1H, m), 6.69 (1H, d), 6.74 (1H, d), 7.30-7.39 (3H, m), 7.50 (1H, d)

### Example 10

### 3-Chloro-N-{(3S)-1-[1-(methylamino)-2,3-dihydro-1H-inden-5-yl]-2-oxo-3-pyrrolidinyl}-1H-indole-6-sulfonamide

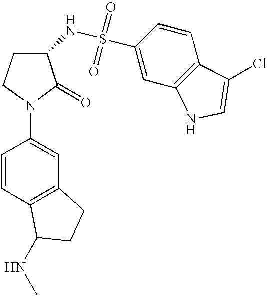

The title compound was prepared from Intermediate 19 using the procedure described for Example 8.

Mass spectrum: Found: MH⁺ 457

H.p.l.c. Rₜ 2.42 min

¹H nmr (DMSO-d₆) δ: 1.68 (2H, m), 2.02 (1H, m), 2.23 (1H, m), 2.27 (3H, d), 2.70 (1H, m), 2.88 (1H, m), 3.60 (2H, m), 3.98 (1H, t), 4.18 (1H, m), 7.27 (1H, d), 7.32-7.43 (2H, m), 7.63 (2H, m), 7.82 (1H, s), 7.98 (1H, s), 11.91 (1H, brs, NH)

### Example 11

### N-[(3S)-1-(1-Amino-2,3-dihydro-1H-inden-5-yl)-2-oxo-3-pyrrolidinyl]-6-chloro-2-naphthalenesulfonamide

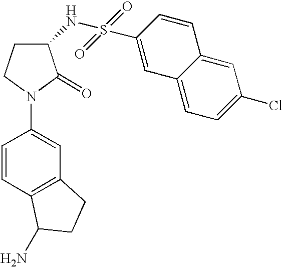

A suspension of N-[5-((3S)-3-{[(3-chloro-1H-indol-6-yl)sulfonyl]amino}-2-oxo-1-pyrrolidinyl)-2,3-dihydro-1H-inden-1-yl]-2,2,2-trifluoro-N-methylacetamide (see Intermediate 23) (158 mg) in MeOH (7 ml) and MeCN (5 ml) was treated with a solution of sodium carbonate (303 mg) in water (2.45 ml) and the suspension was stirred at. 40° C. under nitrogen for 24 h. The reaction mixture was evaporated to dryness and mixed with water (10 ml) and MeOH (10 ml) and treated with sodium carbonate (150 mg) and cloudy mixture was heated at 40° C. for 3 h. The reaction was still incomplete, however the reaction mixture was evaporated to dryness and purified on a 6 g Oasis® column eluting with [95:5] water:MeCN to 100% MeCN. The product fractions were combined and purified on a 20 g SCX column eluting with MeOH to 2N ammonia in MeOH to give the title compound as a cream solid. (71.1 mg).

Mass spectrum: Found: MH⁺ 454

H.p.l.c. Rₜ 2.58 min

¹H nmr (DMSO-d₆) δ: 1.56 (1H, m), 1.71 (1H, m), 2.15 (1H, m), 2.32 (1H, m), 2.66 (1H, m), 2.81 (1H, m), 3.60 (2H, m), 0.15 (1H, t), 4.28 (1H, m), 7.27-7.37 (3H, m), 7.69 (1H, m), 7.96 (1H, m), 8.12 (1H, d), 8.21 (2H, m), 8.55 (1H, s)

### Example 12

### 6-Chloro-N-methyl-N-{(3S)-1-[1-(methylamino)-2,3-dihydro-1H-inden-5-yl]-2-oxo-3-pyrrolidinyl}-2-naphthalenesulfonamide

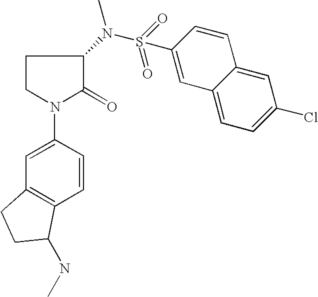

A solution of N-(5-{(3S)-3-[[(6-chloro-2-naphthalenyl)sulfonyl](methyl)amino]-2-oxo-1-pyrrolidinyl}-2,3-dihydro-1H-inden-1-yl)-2,2,2-trifluoro-N-methylacetamide (see Intermediate 24) (113 mg) in MeOH (5 ml) and water (1 ml) was treated with potassium carbonate (125 mg). The reaction was stirred at room temperature for 22 h then evaporated to dryness and partitioned between water (10 ml) and ethyl acetate (20 ml). The layers were separated and the organic extract was passed through a hydrophobic frit containing solid magnesium sulphate and evaporated to dryness and purification on a 5 g SCX column eluting with DCM: MeOH to 10% ammonia in MeOH gave the title compound as white foam (50 mg).

Mass spectrum: Found: MH⁺ 484

H.p.l.c. Rₜ 2.73 min

¹H nmr (DMSO-d₆) δ: 1.72 (1H, m), 1.97 (1H, m), 2.15 (1H, m), 2.23 (1H, m), 2.27 (3H, s), 2.70 (1H, m), 2.76 (3H, s), 2.87 (1H, m), 3.64 (1H, t), 3.75 (1H, q), 3.98 (1H, t), 5.06 (1H, t), 7.26 (1H, d), 7.33 (1H, m), 7.43 (1H, s), 7.71 (1H, dd), 7.96 (1H, dd), 8.13 (1H, d), 8.23 (2H, m), 8.59, (1H, s)

In Vitro Assay for Inhibition of Factor Xa

Compounds of the present invention were tested for their Factor Xa inhibitory activity as determined in vitro by their ability to inhibit human Factor Xa in a fluorogenic assay, using Rhodamine 110, bis-CBZ-glycylglycyl-L-arginine amide as the fluorogenic substrate. Compounds were diluted from a 10 mM stock solution in dimethylsulfoxide at appropriate concentrations. Assay was performed at room temperature using buffer consisting of: 50 mM Tris-HCl, 150 mM NaCl, 5 mM CaCl₂, pH 7.4 containing human Factor Xa (final concentration of 0.0003 U.ml-1). Compound and enzyme were preincubated for 15 min prior to addition of the substrate (final concentration of 10 μM). The reaction was stopped after 3 hrs with the addition of H-D-Phe-Pro-Arg-Chloromethylketone. An LJL-Analyst fluorimeter was used to monitor fluorescence with 485 nm excitation/535 nm emission. To obtain IC₅₀ values the data were analysed using ActivityBase® and XLfit®.

Calculation of Ki values:

Ki=IC₅₀/(1+[Substrate]/Km)

The Ki value for the above assay can be obtained by dividing the IC₅₀ value by 1.6.

All of the synthetic Example compounds tested by the above described in vitro assay were found to exhibit Factor Xa inhibitory activity (Examples 1-12). Preferably, compounds have a Ki value of less than 1M (Examples 1-12). More preferably, compounds have a Ki value of less than 0.1 μM (Examples 1-12). Most preferably, compounds have a Ki value of less than 50 nM (Examples 1, 2, 3, 4, 5, 7, 8, 10, 11, 12).

Method for Measurement of Prothrombin Time (PT)

Blood was collected into a sodium citrate solution (ratio 9:1) to give a final concentration of 0.38% citrate. Plasma was generated by centrifugation of citrated blood samples at 1200×g for 20 min at 4° C. and stored at −20° C. until use. PT analysis was conducted using plasma pooled from 4 separate donors (2 male and 2 female).

The PT test was performed using the BCS Coagulation Analyzer (Dade Behring). For assay, 50 ul of plasma containing test compound at concentrations ranging from 0.03 to 100 uM (made from a 100 uM stock containing 1% DMSO in plasma) was combined with 100 ul of Thromboplastin C Plus (Dade Behring). Upon addition of the reagents, absorbance at 405 nm was monitored and time to clot formation is determined (normal range for human plasma is 10.6-12.4 seconds).

All of the synthetic Example compounds tested by the above described assay were found to exhibit activity (Examples 1-11).

General Purification and Analytical Methods

LC/MS Method

Analytical HPLC was conducted on a Supelcosil LCABZ+PLUS column (3 μm, 3.3 cm×4.6 mm ID) eluting with 0.1% HCO₂H and 0.01M ammonium acetate in water (solvent A), and 95% MeCN and 0.05% HCO₂H in water (solvent B), using the following elution gradient 0-0.7 min 0% B, 0.7-4.2 min 0→100% B, 4.2-5.3 min 100% B, 5.3-5.5 min 100→0% B at a flow rate of 3 ml/min (System 1). The mass spectra (MS) were recorded on a Fisons VG Platform mass spectrometer using electrospray positive ionisation [(ES+ve to give MH⁺ and M(NH₄)⁺ molecular ions] or electrospray negative ionisation [(ES-ve to give (M-H)⁻ molecular ion] modes.

¹H nmr spectra were recorded using a Bruker DPX 400 MHz spectrometer using tetramethylsilane as the external standard.

Biotage™ chromatography refers to purification carried out using equipment sold by Dyax Corporation (either the Flash 40i or Flash 150i) and cartridges pre-packed with KPSil™.

Mass directed preparative h.p.l.c. refers to methods where the material was purified by high performance liquid chromatography on a HPLCABZ+ 5 μm column (5 cm×10 mm internal diameter) with 0.1% HCO₂H in water and 95% MeCN, 5% water (0.5% HCO₂H) utilising the following gradient elution conditions: 0-1.0 min 5% B, 1.0-8.0 min 5→30% B, 8.0-8.9 min 30% B, 8.9-9.0 min 30→95% B, 9.0-9.9 min 95% B, 9.9-10 min 95→0% B at a flow rate of 8 ml min⁻¹ (System 2). The Gilson 202-fraction collector was triggered by a VG Platform Mass Spectrometer on detecting the mass of interest.

Hydrophobic frits refers to filtration tubes sold by Whatman.

SPE (solid phase extraction) refers to the use of cartridges sold by International Sorbent Technology Ltd. Silica SPE and SCX SPE were used.

Combi Flash® Companion™ refers to an automated purification system sold by ISCO Inc.

Oasis® columns refers to the use of liquid phase extraction cartridges sold by Waters.

The internal dimensions of the Chiralpak AD column is 0.46×25 cm.

What is claimed is:

1. At least one chemical entity chosen from compounds of formula (I): 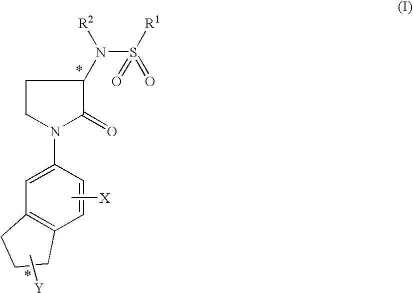
wherein:
R¹ represents a group selected from: 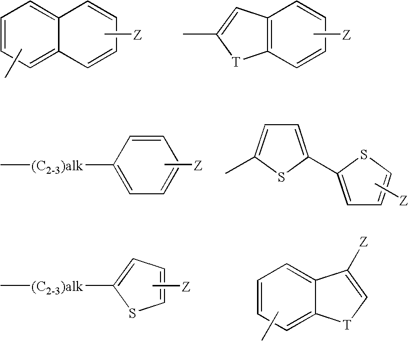
each ring of which optionally contains a further heteroatom N,
Z represents an optional substituent halogen,
alk represents alkylene or alkenylene,
T represents S, O or NH;
R² represents hydrogen, —C₁₋₆alkyl, —C₁₋₃alkylCONR^{a}R^{b}, —C₁₋₃alkylCO₂C₁₋₄alkyl, —CO₂C₁₋₄alkyl or —C₁₋₃alkylCO₂H;
R^{a} and R^{b} independently represent hydrogen, —C₁₋₆alkyl, or together with the N atom to which they are bonded form a 5-, 6- or 7-membered non-aromatic heterocyclic ring optionally containing an additional heteroatom selected from O, N and S, optionally substituted by C₁₋₄alkyl, and optionally the S heteroatom is substituted by O, i.e. represents S(O)ₙ;
n represents 0-2;
X represents an optional substituent on the indane ring selected from halogen, —C₁₋₄alkyl, —C₂₋₄alkenyl and —CF₃;
Y represents a group —(CH₂)ₘNR^{c}R^{d} substituted on the non-aromatic portion of the indane ring;
R^{c} and R^{d} independently represent hydrogen, —C₁₋₆alkyl, —C₁₋₄alkylOH, or together with the N atom to which they are bonded form a 4-, 5-, 6- or 7-membered non-aromatic heterocyclic ring, the 5-, 6- or 7-membered non-aromatic heterocyclic ring optionally containing an additional heteroatom selected from O, N or S, and the 4-, 5-, 6- or 7-membered non-aromatic heterocyclic ring optionally substituted by C₁₋₄alkyl or halogen;
m represents 0-2;
and pharmaceutically acceptable derivative(s) thereof.

2. At least one chemical entity according to claim 1 wherein:
R¹ represents a group selected from: 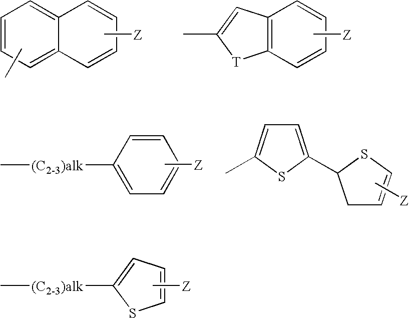
each ring of which optionally contains a further heteroatom N,
Z represents an optional substituent halogen,
alk represents alkylene or alkenylene,
T represents S, O or NH.

3. At least one chemical entity according to claim 1 wherein R¹ represents a group selected from: 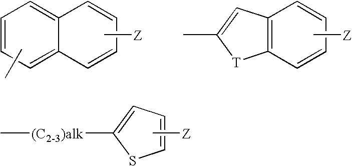
each ring of which optionally contains a further heteroatom N,
Z represents an optional substituent halogen,
alk represents alkylene or alkenylene,
T represents S, O or NH.

4. At least one chemical entity according to claim 1 wherein R² represents hydrogen.

5. At least one chemical entity according to claim 1 wherein X represents an optional substituent selected from: halogen and —C₁₋₄alkyl.

6. At least one chemical entity according to claim 1 wherein Y represents a group —NR^{c}R^{d}.

7. At least one chemical entity according to claim 1 selected from:
6-Chloro-N-{1-[1-(dimethylamino)-2,3-dihydro-1H-inden-5-yl]-2-oxo-3-pyrrolidinyl}-2-naphthalenesulfonamide;
(E)-2-(5-Chloro-2-thienyl)-N-{1-[1-(dimethylamino)-2,3-dihydro-1H-inden-5-yl]-2-oxo-3-pyrrolidinyl}ethenesulfonamide;
6-Chloro-N-{1-[1-(dimethylamino)-2,3-dihydro-1H-inden-5-yl]-2-oxo-3-pyrrolidinyl}-1-benzothiophene-2-sulfonamide;
6-Chloro-N-{(3S)-1-[-(dimethylamino)-2,3-dihydro-1H-inden-5-yl]-2-oxo-3-pyrrolidinyl-2-naphthalenesulfonamide;
3-Chloro-N-{(3S)-1-[1-(dimethylamino)-2,3-dihydro-1H-inden-5-yl]-2-oxo-3-pyrrolidinyl}-1H-indole-6-sulfonamide;
2-(5-Chloro-2-thienyl)-N-{(3S)-1-[1-(dimethylamino)-2,3-dihydro-1H-inden-5-yl]-2-oxo-3-pyrrolidinyl}ethanesulfonamide;
6-Chloro-N-{(3S)-1-[1-(methylamino)-2,3-dihydro-1H-inden-5-yl]-2-oxo-3-pyrrolidinyl}-2-naphthalenesulfonamide;
(E)-2-(5-Chloro-2-thienyl)-N-{(3S)-1-[1-(methylamino)-2,3-dihydro-1H-inden-5-yl]-2-oxo-3-pyrrolidinyl}ethenesulfonamide;
2-(5-Chloro-2-thienyl)-N-{(3S)-1-[1-(methylamino)-2,3-dihydro-1H-inden-5-yl]-2-oxo-3-pyrrolidinyl}ethanesulfonamide;
3-Chloro-N-{(3S)-1-[1-(methylamino)-2,3-dihydro-1H-inden-5-yl]-2-oxo-3-pyrrolidinyl}-1H-indole-6-sulfonamide;
N-[(3S)-1-(1-Amino-2,3-dihydro-1H-inden-5-yl)-2-oxo-3-pyrrolidinyl]-6-chloro-2-naphthalenesulfonamide;
6-Chloro-N-methyl-N-{(3S)-1-[-(methylamino)-2,3-dihydro-1H-inden-5-yl]-2-oxo-3-pyrrolidinyl}-2-naphthalenesulfonamide;
and pharmaceutically acceptable derivatives thereof.

8. (canceled)

9. A pharmaceutical composition comprising at least one chemical entity according to claim 1 together with at least one pharmaceutical carrier and/or excipient.

10. (canceled)

11. A method of treating a patient suffering from a condition susceptible to amelioration by a Factor Xa inhibitor comprising administering a therapeutically effective amount of at least one chemical entity according to claim 1.

12. A process for preparing a compound(s) of formula (I) which comprises reacting compound(s) of formula (II) or an acid addition salt thereof with compound(s) of formula (III): 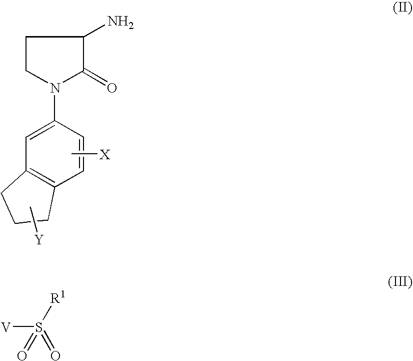
wherein V is a suitable leaving group and Y is a protected or unprotected group.
